(19) United States
(12) Patent Application Publication
(10) Pub. No.: US 2026/0232876 A1
(43) Pub. Date: Aug. 13, 2026

(54) POROUS METAL STRUCTURES INCORPORATING NANOPARTICLES

(71) Applicant: Yissum Research Development Company of the Hebrew University of Jerusalem Ltd., Jerusalem (IL)

(72) Inventors: Ofra BENNY, Jerusalem (IL); Chalom ZEMMOUR, Jerusalem (IL)

(73) Assignee: Yissum Research Development Company of the Hebrew University of Jerusalem Ltd., Jerusalem (IL)

(21) Appl. No.: 19/471,429

(22) PCT Filed: May 2, 2024
(86) PCT No.: PCT/IL2024/050414

§ 371 Date: Oct. 1, 2025

Related U.S. Application Data
(60) Provisional application No. 63/463,654, filed on May 3, 2023.

(51) Int. Cl. A61L 31/14; A61L 31/02; A61L 31/10; A61L 31/16; C23F 1/14

(52) U.S. Cl. CPC A61L 31/146; A61L 31/022; A61L 31/10; A61L 31/148; A61L 31/16; C23F 1/14

(57) ABSTRACT

The invention generally concerns etched metallic surface containing nanoparticle-loaded nanopores.

### TECHNOLOGICAL FIELD

The invention generally contemplates porous metal structures incorporating nanoparticles and uses thereof.

### BACKGROUND

Porous metals are a class of materials with unique properties, including high surface area, high permeability, tunable pore size and pore distribution. These properties make them suitable for a wide range of applications, including catalysis, filtration, energy storage, and biomedical engineering. Porous metal surfaces increasingly gain interest due to their superior properties compared with other porous materials such as polymers or ceramics in terms of their mechanical stability, oxidation resistance, lightweight, and additional physical properties, e.g., their electrical, acoustic, thermal, and vibration properties.

The high surface area of porous metals allows a higher number of chemical reactions to occur at active sites, making them appropriate for catalytic applications. In filtration applications, such as gas separation and water purification, the tunable pore size and distribution make porous metals useful for separating different-sized particles. Porous metals also have potential in energy storage applications, such as batteries and supercapacitors. In the biomedical field, porous metals are used for bone implants, as they can promote bone tissue regeneration and integration. Some examples of porous metals include titanium, nickel, and stainless-steel foams, which are widely used in industrial and biomedical applications. Overall, the unique properties of porous metals make them versatile and promising materials for various fields The porosity of the surface may provide important advantages in the integration of active molecules; environmental factors such as temperature, light, or humidity can alter the stability of active molecules. Depositing molecules inside pores may protect them and potentially improve stability and shelf life. The release rate of the active material can be controlled by several factors, including the size and shape of the pores, the surface chemistry of the porous material, and the interaction between the active compound and the porous material. Shifting from dense to porous materials increases the effective surface area available for interactions between the active components and the matrix. Therefore, it may serve as an important strategy for improving the stability, controlled release, and efficacy of various porous devices.

There are various methods for producing porous metals, including powder metallurgy, foaming, chemical vapor deposition (CVD), electrodeposition, and chemical etching. In powder metallurgy, a mixture of metal powders and filler materials is sintered and then removed to create pores. Foaming involves creating a metallic foam by mixing a molten metal with a blowing agent and cooling it rapidly. In CVD, metal atoms are deposited onto a substrate to create a porous structure and in electrodeposition, metal ions are reduced at the cathode to form a metal layer with controlled porosity. These methods hold significant advantages in controlling the pores' size and shape and having the ability to produce complex shapes. However, all these processes require special equipment, the procedures may be relatively slow, not compatible for large surfaces, and may result in products having limited mechanical strengths.

### GENERAL DESCRIPTION

The inventors of the technology disclosed herein have developed a process for treating already-formed metallic objects to effectively and simply generate surface pores that may be treated to entrap a variety of particulate materials. As demonstrated, the process does not involve use of complex equipment and is compatible for large surfaces and products having limited mechanical strengths.

Despite the many advantages associated with porous metals in fields such as biomedicine, electronics, and energy, one of the major challenges in utilizing porous metals is to incorporate active compounds, either small molecules or macromolecules, on these surfaces. Coatings that contain active molecules have previously been used for biomedical applications to enable the slow release of drugs, e.g., with drug-eluting cardiovascular stents. However, direct deposition of organic materials on metals by coatings is very difficult due to the challenge of obtaining uniform coatings, as well as issues relating to layer adherence and mechanical stability.

In a study leading to the present application, a process for manufacturing a great variety of different porous metals, e.g., aluminum, gold, and titanium, was developed. Pertinent physicochemical measurements were carried out to characterize the porous surfaces. Following the production of the porous metal surfaces, a new methodology was developed for incorporating particles of active or functional materials into pores formed in the metals surface by using mechanical entrapment of nanoparticles. The novel methodology not only enables incorporation of nanoparticles of different sizes, morphologies and compositions, which may be used for controlled release of materials, but can also be used to modify or modulate surface properties of pre-formed metals, or objects formed therefrom, without needing to resort to metallurgic processes, which typically involve high temperature conditions and potentially impose changes to the material composition.

In its broadest scope, the invention provides an object having a metallic surface with an etched surface porosity of a predefined porosity profile, wherein the etched surface porosity comprises a plurality of etched nanopores comprising or containing nanoparticles of a material different from the metal forming the surface.

The invention provides an object having a metallic surface with a chemically etched surface porosity comprising a plurality of etched surface nanopores, said plurality of nanopores being of a predefined porosity profile and containing nanoparticles of a material different from the metal forming the surface.

Also provided is an object having a metallic surface with a surface porosity of a predefined porosity profile, wherein the surface porosity comprising a plurality of chemically etched surface nanopores, at least a portion of which or each comprising, holding, docking, encapsulating, incorporating or hosting nanoparticles of a material different from the metal forming the surface.

Advantageously, the “object” of the invention is an “article of manufacture” having a metallic surface that is treated, in accordance with the present invention, to generate a plurality of nanopores in the metallic surface. The object may be a pre-formed object that has been fully manufactured, but for its surface porosity. As the porosity is achievable by chemical etching, as disclosed herein, the object may be of any size and shape and is not limited by any mechanical, physical or chemical factor. The object may be a metallic object or an object that is non-metallic but having a metallic surface. The ability to generate nanopores of predetermined characteristics, i.e., with a predefined porosity profile, allows to selectively generate nanopores on any pre-formed object having a metal surface, at any stage following object production, and potentially following object commercialization.

Chemical etching of the metallic surface provides an etched surface porosity of predefined characteristics. In other words, the etching protocol (i.e., conditions used for achieving etching of the metallic surface, wherein the etching induces pore formations in the metallic surface) may be tailored or varied to obtain a metallic surface with pores of predefined pore sizes, pore densities, pore distribution and/or pore concentration (number of pores per surface area). As the etching protocol may be finely tuned, the predefined pore sizes, pore densities, pore distribution and/or a pore concentration (number of pores per surface area), each alone or in combination with another, defining a porosity profile, may vary between objects and even between different regions of the same metallic surface, thereby rendering the surface suitable for hosting a variety of different nanoparticles or nanoparticle populations.

Pore formation according to the present invention is limited to formation by chemical etching and to porous metallic surfaces achievable by chemical etching. Metallic surfaces having pore structures formed by lithography, material deposition or material removal, by sintering, by using porous materials or by any other way other than chemical etching are excluded. Unlike any of these methodologies, chemical etching for forming pores in the metallic surface provides a versatile and cost-effective method for fabricating porous metals with controllable porosity profiles. The selection of metals is unlimited, wherein each solid metallic surface may be treated under predefined etching conditions to obtain a finely calibrated and designed porosity profile, meeting one or another intended use.

The object of the invention may be any object, formed of any material, and having at least one metallic surface region, namely a surface region formed of a metal, a metal alloy, a metal oxide or a metal containing composite material. The metallic surface may be of a solid metal such as aluminum, gold, silver, titanium, tungsten, copper, nickel, iron, chromium, tin, and others. Similarly, the metallic surface may be formed of a metal alloy such as aluminum alloys, copper alloys, brasses and bronzes, cast iron, nickel alloys and superalloys, stainless steel, high carbon steel and low carbon steel, and others. The objects may be fully metallic or may comprise a surface film or a surface layer of an etched metal according to the invention. The etching may be achieved at any stage after the object has been manufactured or after the metallic film or metallic layer has been manufactured. Thus, generally, objects undergoing pore patterning by chemical etching are preformed prior to etching.

As used herein, the term “chemical etching”, or simply etching, refers to a process of forming nanopores or nanocavities in the metallic surface by contacting the surface with an etchant or an etching solution, as further disclosed herein. The term does not include mechanical etching or light mediated etching. The expressions “etched surface porosity”, or “etched surface nanopores”, or “chemically etched metallic surface”, or “chemically etched surface nanopores” are interchangeable and refer to objects wherein their metallic surface has been chemically etched to provide the nanopores. While objects of the invention may further include patterned regions fabricated by means other than chemical etching, these fabrication methods and patterns do not constitute part of the invention.

The pores formed in the metallic surface are nanopores. They are typically of a diameter or a size (e.g., length, width, diameter, etc.) that is nanometric in size, namely being between 10 and 500 nm. The nanopores are not necessarily channels, or passages that run through the width or thickness of the object or the surface. The nanopores may be discrete nanoscale openings or hollows or cavities, having side and back walls and a front opening permitting docking of nanoparticles. The pores inner walls may not be smooth or polished. The inner walls surface roughness may vary and may assist in anchoring the nanoparticles in their position.

As noted, the nanopores are nanometric in size, having at least one dimension, or more than one dimension, or all measurable dimensions that are between 10 and 500 nm. In some embodiments, the pores average size (diameter) is between 10 and 500, 10 and 400, 10 and 300, 10 and 200, 10 and 100, 10 and 90, 10 and 80, 10 and 70, 10 and 60, 10 and 50, 10 and 40, 10 and 30, 10 and 20, 15 and 200, 15 and 150, 15 and 100, 15 and 95, 15 and 90, 30 and 150, 40 and 150, 50 and 150, 60 and 150, 70 and 150 or between 50 and 500 nm.

The depth of the pores may also be nanometric in size. The depth of the pores is sufficiently large to permit stable docking of the nanoparticles therein. The depth size may be similarly selected as above between 10 and 500 nm.

The nanopores' dimension may be determined by any spectroscopic method known in the art. In some non-limiting embodiments, the methodologies used for determining the nanopores' sizes (and shapes, where desired) is scanning electron microscopy (SEM) or transmission electron microscopy (TEM), as further demonstrated herein.

In some embodiments, the pores average size may be pre-designed or determined based on the etchant used, its concentration and time of exposure, as further described hereinbelow.

The pores density, namely the number of pores per area of the metallic surface, may vary. Typically, the surface may comprise between 20 and 1000 pores per square micrometer (pores/μm²). The actual number pf pores per surface area may vary by ±20% of any indicated value.

In some embodiments, the pore density is between 20 and 900, 20 and 800, 20 and 700, 20 and 600, 20 and 500, 20 and 400, 20 and 300, 20 and 200, 20 and 100, 25 and 750, 25 and 650, 25 and 550, 25 and 450, 25 and 350, 25 and 250, 25 and 150, 25 and 50, 100 and 800, 100 and 700, 100 and 600, 100 and 500, 100 and 400, 100 and 300, 100 and 200 or between 500 and 800 pores/μm².

In some embodiments, the metallic surface is aluminum having a pore density of between 20 and 1000, 20 and 900, 20 and 800, 20 and 700, 25 and 750, 100 and 800, 100 and 750, 550 and 750, 650 and 750, 700 and 800 or about 750 pores/μm².

In some embodiments, the metallic surface is gold having a pore density of between 20 and 1000, 20 and 400, 20 and 300, 20 and 200, 20 and 100, 25 and 350, 25 and 250, 25 and 150, 100 and 800, 100 and 700, 100 and 600, 100 and 500, 100 and 400, 100 and 300, 100 and 200, 90 and 250, 90 and 200, 90 and 150, 90 and 100 or about 100 pores/μm²In some embodiments, the metallic surface is titanium having a pore density of between 20 and 1000, 20 and 100, 20 and 90, 20 and 80, 20 and 70, 20 and 60, 20 and 50, 20 and 40, 20 and 30, 25 and 65, 25 and 55, 25 and 45, 25 and 35 or about 25 pores/μm².

Etching enables formation and patterning of the pores on the surface such that the pores may decorate only certain regions of the surface or define regions of greater pore densities. Thus, the pores may not be evenly formed over the full metallic surface. In some cases, for certain applications, the pores are formed substantially evenly on the surface. In other cases, the pores may be formed on predefined surface regions. Similarly, in some cases, the pores share a similar or identical porosity profile, while in other cases, the pores may differ from each other in size, shape, distribution, density, etc.

The invention further provides a metallic surface having a plurality of nanopores provided in a pore pattern formed by chemical etching of the metallic surface, the plurality of chemically etched nanopores comprising, holding, docking or hosting nanoparticles of a material different from the metal forming the surface. In some embodiments, the metallic surface is a surface of a metallic object. In other embodiments, the metallic surface is a surface of a non-metallic object.

As stated herein, nanoparticles occupy the nanopores of the metallic object or surface. The concentration of the nanoparticles depends, inter alia, on the pore concentration (the number of pores) or pore density (number of pores per surface area). Typically, the nanoparticles occupy only the nanopores and are not associated to a non-porous region of the metallic surface. In other words, the nanoparticles are solely provided in the etching-formed pores. The degree of nanoparticle docking in the nanopores may vary and depends on a variety of factors, including the intended use, the pore concentration and the type and size of the nanoparticles. Loading of the nanopores may be limited or maximal. In other words, in some cases between 1 and 100% of the nanopores are loaded with the nanoparticles. In some embodiments, all or substantially all of the nanopores are loaded with nanoparticles. In some embodiments, at least 1, 10, 20, 30, 40, 50, 60, 70, 80, or 90% of the nanopores are loaded. In some embodiments, between 1 and 10, or 5 and 15, or 10 and 20, or 15 and 25, or 20 and 30, or 25 and 35, or 30 and 40, or 35 and 54, or 40 and 45, or 45 and 50, or 50 and 60, or 55 and 65, or 60 and 70, or 65 and 75, or 70 and 80, or 75 and 85, or 80 and 90, or 85 and 95, or 90 and 100% of the nanopores are loaded with nanoparticles according to the invention.

The nanoparticles contained within the nanopores require no physical or chemical attachment to the pore walls. It is believed that their docking in the nanopores is favored due to possible stabilizing interactions between the metal walls of the nanopores and the nanoparticle surface. The rough internal surface of the nanopores assists in their secure entrapment. Therefore, the metallic object or surface having the nanoparticles-loaded nanopores are provided uncoated, such that the nanoparticles surface is surface-exposed and is thus capable of undergoing mechanical or chemical degradation over time. Depending on the nanoparticles' material, the degradation of the nanoparticles, e.g., to release their content, may be immediate or occur over time, thereby permitting a tailored degradation and release profiles.

In some cases, however, the metallic object or surface is provided with a protective external coating or film that prevents premature, unwanted or accidental degradation or release of the nanoparticles from the nanopores. The coating or film may be formed of a degradable or a biodegradable material that degrades over time to expose the nanoparticle-loaded nanopores of the metallic object or surface. The degradable or biodegradable material of the external coating or film may be selected amongst cellulose, starch, polyhydroxyalkanoates, polylactide, polycaprolactone, collagen, polyesters, polycarbonates, polypeptides and others. The thickness of the film or coating may be between 10 nm and 5 microns.

In some embodiments, the thickness of the external coating or film is between 10 and 900, or 10 and 800, or 10 and 700, or 10 and 600, or 10 and 500, or 10 and 400, or 10 and 300, or 10 and 200, or 10 and 100, or 10 and 90, or 10 and 80, or 10 and 70, or 10 and 60, or 10 and 50, or 100 and 900, or 100 and 800, or 100 and 700, or 100 and 600, or 100 and 500, or 100 and 600, or 100 and 500, or 100 and 400, or 100 and 300, or 100 and 200, or 900 nm and 1 microns, or 900 nm and 1.5 microns, or 900 nm and 2 microns, or 900 nm and 2.5 microns, or 900 nm and 3 microns, or 900 nm and 3.5 microns, or 900 nm and 4 microns, or 900 nm and 4.5 microns, or 900 nm and 5 microns, or 1 and 1.5 microns, or 1 and 2 microns, or 1 and 2.5 microns, or 1 and 3 microns, or 1 and 3.5 microns, or 1 and 4 microns, or 1 and 4.5 microns, or between 1 and 5 microns.

The nanoparticles loaded into nanopores of the metallic object or surface are selected amongst functional particles having nanometric dimensions. Clearly, the nanoparticles are selected to be of a size capable of entering and docking within the nanopores and are further selected from materials endowing the metallic object or surface with functional and/or mechanical properties. As such, the nanoparticles should be of a nanometric size, a dimension or a diameter that is smaller than the average size of the nanopores to receive the nanoparticles. The size of the nanoparticles is between 1 and 10% smaller (in size or diameter) than the average size or diameter of the nanopores selected to receive the nanoparticles. For example, where the nanopores are of an average size of 50 nm, the average size of the nanoparticles should be between 49.5 and 45 nm.

The nanoparticles are generally spheroidal or spherical nanostructures, while less symmetric or other non-spherical shapes may also be used. The nanoparticles may be selected from nanocarriers, nanospheres, nanocapsules, core/shell nanostructures, micelles, matrix nanostructures formed of a matrix material such as a polymer, a metal, etc., and any other type of a nanoparticle, as known in the art. The nanoparticles may be formed of a single material or may be in a form capable of holding or containing a second or a further material. The type of nanoparticles used may be selected based on the intended use of the metallic object or surface. For example, where the object is intended for delivery or release of a material, e.g., for use in pharma or cosmetics, for releasing a material over time, the nanoparticles may be in a form of a nanocarrier, a core/shell nanostructure or a matrix nanoparticle containing the material to be released. Similalrly, where the nanoparticles are intended to modulate a mechanical, physical, chemical or a functional property of the metallic object or surface, the nanoparticles may be selected amongst such nanoparticles having or contributing to the intended property. Such may be conductive nanoparticles, insulator nanoparticles, metallic nanoparticles, magnetic nanoparticles, catalyst nanoparticles, optically-active nanoparticles, hard or soft nanoparticles and others.

Thus, in some embodiments, the nanoparticles may be selected from:
nanoparticles of a first material encapsulating or containing a second material (wherein the second material may be, e.g., active materials such as drugs, cosmetic materials, fragrant materials, coloring materials, flavoring materials, antimicrobial materials (including antibacterial, antiviral, antifungal, etc), insecticidal materials, insect-repelling materials, agriculturally acceptable materials (including antibacterial, antiviral, antifungal materials, etc), and others, wherein the at least one active material is to be released from the nanoparticles over time);
nanoparticles of a material or a material composition having a predefined property; the nanoparticles may be metallic nanoparticles, conductive nanoparticles, insulator nanoparticles, magnetic nanoparticles (ferromagnetic, paramagnetic, etc), catalyst nanoparticles, optically-active nanoparticles, hard or soft nanoparticles, and others;
polymeric nanoparticles of a degradable or a biodegradable polymer;
polymeric nanoparticles of a degradable or biodegradable polymer containing at least one active material selected from drugs, cosmetic materials, fragrant materials, coloring materials, flavoring materials, antimicrobial materials (including antibacterial, antiviral, antifungal, etc), insecticidal materials, insect-repelling materials, agriculturally acceptable materials (including antibacterial, antiviral, antifungal materials, etc), and others, wherein the at least one active material is to be released from the nanoparticles over time;
non-polymeric nanoparticles of a degradable or biodegradable material containing at least one active material selected from drugs, cosmetic materials, fragrant materials, coloring materials, flavoring materials, antimicrobial materials (including antibacterial, antiviral, antifungal, etc), insecticidal materials, insect-repelling materials, agriculturally acceptable materials (including antibacterial, antiviral, antifungal materials, etc), and others, wherein the at least one active material is to be released from the nanoparticles over time;
polymeric nanoparticles of a degradable or biodegradable polymer containing at least one active material selected from drugs, cosmetic materials, fragrant materials, coloring materials, flavoring materials, antimicrobial materials (including antibacterial, antiviral, antifungal, etc), insecticidal materials, insect-repelling materials, agriculturally acceptable materials (including antibacterial, antiviral, antifungal materials, etc), and others, wherein the at least one active material is to be released from the nanoparticles overtime, and wherein the degradable or biodegradable polymer is a functional material having one or more property, such as magnetic, conductive, etc;
non-polymeric nanoparticles of a degradable or biodegradable material containing at least one active material selected from drugs, cosmetic materials, fragrant materials, coloring materials, flavoring materials, antimicrobial materials (including antibacterial, antiviral, antifungal, etc), insecticidal materials, insect-repelling materials, agriculturally acceptable materials (including antibacterial, antiviral, antifungal materials, etc), and others, wherein the at least one active material is to be released from the nanoparticles over time, and wherein the degradable or biodegradable material is a functional material having one or more property, such as magnetic, conductive, etc; and
any other nanoparticle.

In some embodiments, the nanoparticles are polymeric nanoparticles formed from different types of natural and synthetic polymers having bio/degradability. Such polymeric nanoparticles may be used in applications requiring improved or increased stability in various microenvironments, slow release of drugs due to the polymer degradation, and their diversity in the types of active materials that can be contained or encapsulated therein. The polymer may be selected amongst homopolymers, copolymers, terpolymer, block copolymers, grafted polymers and the like. Such polymers may be selected amongst polyolefins, poly acrylates and methacrylates, styrene polymers, polyesters, polyamides, polyimines, polycarbonates, natural polymers, cellulosic materials, polysaccharides, thermoplastic elastomers, polyvinyl alcohols, polynitriles, polyacetals, polyimides, polyarylketones, polyetherketones, polyhydroxyalkanoates, polycaprolactones, polyurethanes, polysulfones, polyphenylene oxides, polyphenylene sulfides, polyacetates, liquid crystal polymers, fluoropolymers, ionomeric polymers, thermoplastic elastomers, and blends thereof; wherein the polymer may or may not be degradable or biodegradable.

Examples of suitable polymers include natural polymers such as gelatin, dextran, albumin, chitosan, alginate and others. Synthetic or semi-synthetic polymers may include polylactic acid (PLA), polyglycolic acid (PGA), copolymer of lactic acid and glycolic acid (PLGA), poly (ε-caprolactone) (PCL), polyalkylcyanoacrylate (PACA), poly (ethylene glycol) (PEG), poly (D,L-lactide-co-glycolide) (PLG), polyethyleneimine (PEI), poly (L-lysine), m-PEG-b-PLA, and others.

In some embodiments, the nanoparticles are not polymeric nanoparticles. The non-polymeric nanoparticles may be of an organic material that is not polymeric (such as graphite, graphene, C60 and others), an inorganic material (e.g., silica, titania, alumina, etc), a ceramic material, or a hybrid organic-inorganic material (e.g., a perovskite material, a carbide, etc). The non-polymeric materials may or may not be degradable and may or may not contain an active material to be released.

In some embodiments, the non-polymeric nanoparticles are metallic or metal containing nanoparticles, wherein the metal may be different from the metal of the metallic object or surface (wherein the difference may be in the metal atom, e.g., gold object and iron nanoparticles, or may be in the composition of the metal, e.g., iron object and Fe₃O₄ nanoparticles). The nanoparticles may be metallic nanoparticles selected amongst aluminum, gold, silver, titanium, tungsten, copper, nickel, iron, chromium, tin, and others, or may be metal containing nanoparticles such as metal oxide or metal alloys or a metalloid, such as silicon oxide, titanium oxide, aluminum oxide, iron oxide (e.g., magnetite (Fe₃O₄), maghemite (γ-Fe₃O₂)), and others.

In some embodiments, the object of the invention having a chemically etched metallic surface with a surface porosity of a predefined porosity profile, wherein the surface porosity comprising a plurality of chemically etched surface nanopores comprising, holding, docking or hosting nanoparticles of a material different from the metal forming the surface, wherein the nanoparticles are selected as defined herein, e.g., from:
nanoparticles of a first material encapsulating or containing a second material (wherein the second material is as defined and selected herein, and wherein the second material is optionally to be released from the nanoparticles);
nanoparticles of a material or a material composition having a predefined property; the nanoparticles may be metallic nanoparticles, conductive nanoparticles, insulator nanoparticles, magnetic nanoparticles, catalyst nanoparticles, optically-active nanoparticles, hard or soft nanoparticles, and others;
polymeric nanoparticles of a degradable or a biodegradable polymer;
polymeric nanoparticles of a degradable or biodegradable polymer containing at least one active material selected from drugs, cosmetic materials, fragrant materials, coloring materials, flavoring materials, antimicrobial materials (including antibacterial, antiviral, antifungal, etc), insecticidal materials, insect-repelling materials, agriculturally acceptable materials (including antibacterial, antiviral, antifungal materials, etc), and others, wherein the at least one active material is to be released from the nanoparticles over time;
non-polymeric nanoparticles of a degradable or biodegradable material containing at least one active material selected from drugs, cosmetic materials, fragrant materials, coloring materials, flavoring materials, antimicrobial materials (including antibacterial, antiviral, antifungal, etc), insecticidal materials, insect-repelling materials, agriculturally acceptable materials (including antibacterial, antiviral, antifungal materials, nutrients, fertilizers, etc), and others, wherein the at least one active material is to be released from the nanoparticles overtime;
polymeric nanoparticles of a degradable or biodegradable polymer containing at least one active material selected from drugs, cosmetic materials, fragrant materials, coloring materials, flavoring materials, antimicrobial materials (including antibacterial, antiviral, antifungal, etc), insecticidal materials, insect-repelling materials, agriculturally acceptable materials (including antibacterial, antiviral, antifungal materials, nutrients, fertilizers, etc), and others, wherein the at least one active material is to be released from the nanoparticles over time, and wherein the degradable or biodegradable polymer is a functional material having one or more property, such as magnetic, conductive, etc; and
non-polymeric nanoparticles of a degradable or biodegradable material containing at least one active material selected from drugs, cosmetic materials, fragrant materials, coloring materials, flavoring materials, antimicrobial materials (including antibacterial, antiviral, antifungal, etc), insecticidal materials, insect-repelling materials, agriculturally acceptable materials (including antibacterial, antiviral, antifungal materials, nutrients, fertilizers, etc), and others, wherein the at least one active material is to be released from the nanoparticles over time, and wherein the degradable or biodegradable material is a functional material having one or more property, such as magnetic, conductive, etc.

In some embodiments, the nanoparticles define a single population of nanoparticles or a mixture of two or more different populations of nanoparticles (wherein each population differs from another in the type of nanoparticles, the material composition of the nanoparticles, the size of the nanoparticles, the material contained by the nanoparticles, etc).

Objects and surfaces of the invention, or any article of manufacture formed of or being an object of the invention may be used in a variety of different fields, such as medicine and pharma, electronics, optics, agriculture, chemical catalysis, food industry, diagnosis, thermal insulation, and many others. Thus, objects of the invention may be used for delivery of at least one active material contained in said nanoparticles; or for inducing or modulating or increasing a mechanical property of the metallic object.

The invention thus further provides a device or an object or an implant or an article having a metallic surface having a plurality of nanopores provided in a pore pattern formed by chemical etching of the metallic surface, the plurality of chemically etched nanopores comprising, holding, docking or hosting polymeric nanoparticles comprising at least one pharmaceutical, drug or active agent, wherein the polymeric nanoparticles are configured to degrade or biodegrade overtime to bring about release of the at least one pharmaceutical, drug or active agent.

The at least one pharmaceutical, drug or active agent may be any such material used in medicine or veterinary. The device may be an implant or any medical device positioned against a tissue or an organ of a human or animal subject, e.g., a stent.

The invention further provides a device or an object or an article of manufacture having a metallic surface having a plurality of nanopores provided in a pore pattern formed by chemical etching of the metallic surface, the plurality of chemically etched nanopores comprising, holding, docking or hosting polymeric nanoparticles comprising at least one material, wherein the polymeric nanoparticles are configured to degrade or biodegrade overtime to bring about release of the at least one material.

The at least one material contained in the nanoparticles may be selected from agrochemicals, pharmaceuticals, cosmetic materials, personal care materials, laundering detergents, oral care materials, dental care materials, oil materials, water treatment materials, paint materials, flavoring materials, fragrant materials and other functional materials.

Non-limiting examples of the materials include pigments, dyes, colorants, scale inhibitors, emollient oils, insecticides, detergents, printing inks, corrosion and rust, inhibitors, antioxidants, catalysts, initiators, waxes, dispersants, flame retardants, biocides, anti-fouling agents, odor control agents, cosmetic additives, oxidizing agents, personal care actives, agrochemicals, fertilizers, fats, nutrients, enzymes, natural oils, fragrances, flavor and perfume oils, crop protection agents, medicaments, pharmaceuticals, phase change materials and others.

In some embodiments, the materials may include fragrant materials, insecticidal materials, insect-repelling materials, agriculturally acceptable materials (including antibacterial, antiviral, antifungal materials, nutrients, fertilizers, etc), and others.

The invention further provides an object or an article of manufacture for use in agriculture, the object or article of manufacture having a metallic surface having a plurality of nanopores provided in a pore pattern formed by chemical etching of the metallic surface, the plurality of chemically etched nanopores comprising, holding, docking or hosting polymeric nanoparticles comprising at least one agriculturally acceptable material, wherein the polymeric nanoparticles are configured to degrade or biodegrade overtime to bring about release of the at least one agriculturally acceptable material.

The at least one agriculturally acceptable material may be an insecticidal material, an insect-repelling material, or any other agriculturally acceptable material, including antibacterial, antiviral, antifungal materials, nutrients, fertilizers, growth hormones, herbicides, and others.

Without wishing to be bound by theory, the release of a material from the nanoparticle may depend on a variety of factors including, for example, the type of material, the properties of the material (solid, liquid, boiling point), the conditions of use (exposure to high temperatures, humidity etc), the type of nanoparticle containing the material, the nanoparticle composition (polymeric, inorganic, etc), etc. Typically, the material contained in the nanoparticles may be released slowly from the nanoparticle even before the nanoparticles begin to degrade, or the release may be continuous and increase as the nanoparticle is increasingly degraded. In some cases, the release is temperature or humidity dependent and may not depend or require degradation of the nanoparticle. In other cases, nanoparticle degradation enables release.

The amount released and the rate of release define a controlled release profile which may be designed as a slow release, sustained-release, rapid release, immediate release or designed to release the material a prolonged controlled mode or fashion.

Characteristics of the nanopores and the nanoparticles may be controlled so as to yield a desired controlled release half-life or a desired release over a given period of time. For example, the controlled release of the material contained within the nanoparticle may be over a period of at least about 4 hours (e.g., at least 4, 5, 6, 7, 8, 9, 10, 11, 12, 13, 14 and more hours), or within a period of up to 4 hours (or up to 5, 6, 7, 8, 9, 10, 11, 12, 13, 14 and more hours). In other cases, the release may be over a much longer period of time, e.g., days, weeks or months. For example, less volatile materials and slowly degrading polymeric materials may permit a slow release over a period of 1-30 days, or between 1 and 6 months and longer.

The invention further provides a process for manufacturing an object of the invention, the process comprising contacting a metallic surface of an object having a plurality of surface nanopores with a medium (solution, dispersion or emulsion) comprising nanoparticles and allowing said nanoparticles to occupy at least a portion of the plurality of nanopores, wherein the surface nanopores have been formed by chemical etching.

In some embodiments, the process comprises treating the object to remove nanoparticles present outside of the nanopores (namely on a non-porous surface region of the object).

In some embodiments, the process comprising obtaining the object having an etched metallic surface decorated with a plurality of nanopores.

The process further comprises etching a metallic surface of an object under conditions selected to form a plurality of surface nanopores having a predefined porosity profile (i.e., pore sizes, pore densities, pore distribution and/or a pore concentration (number of pores per surface area)).

The etching conditions selected to form a plurality of surface nanopores typically involve conditions of chemical etching, whereby the metallic surface is contacted or treated with an etchant or an etching solution of a concentration and over a time period sufficient to achieve the desired porosity profile. As disclosed and further demonstrated herein, etching may be achieved by dipping the surface into an etchant or an etching solution, by depositing the etchant or the etching solution onto a surface region of the metallic surface, by dropping small volumes or quantities of the etchant on preselected regions of the surface, or by brushing or spraying or washing the surface with the etchant or etching solution. The deposition may be by any deposition means such as a pipette or by a patterning tool such as a printing device.

The “etchant” is a material used to chemically remove an amount of a metal from a surface region of the metallic surface. The etchant may be used as is, or may be used in a form of an etching solution which comprises one or more etching chemicals and a liquid medium, e.g., water, alcohol, etc. The etchant or etching solution used may be any such material known in the art and may be selected amongst acids and bases, as well as from oxidizing materials. Non-limiting examples of etchants and etching solutions include Adler etchant (comprising copper ammonium chloride, HCl, FeCl₃, water), ammonium persulfate etchant (comprising ammonium persulfate, and water), aluminum Al—NaOH etchant (comprising NaOH and water), etchant commercially available under ASTM No. 30 (comprising ammonia, H₂O₂, and water), etchant commercially available under ASTM No. 97 (comprising KOH and water), etchant commercially available under ASTM No. 157 (comprising CrO₃, HCL and water), Berahas etchant (comprising Na₂S₂O₃, K₂S₂O₅ and water), Carpenters stainless steel etchant (comprising FeCl₃, CuCl₂, HCl, nitric acid, and ethanol), etchant commercially available under Copper No. 1 (comprising nitric acid and water), etchant commercially available under Copper No. 2 (comprising HCl, ferric chloride and water), dichromate etchant (comprising K₂Cr₂O₇, H₂SO₄, NaCl and water), Frys reagent (comprising HCl, copper chloride, water and ethanol), Inconel etchant (comprising nitric acid, HCl, H₂O₂ and water), Kalling's No. 2 (comprising CuCl₂, HCl, and ethanol), waterless Kalling's reagent (comprising CuCl₂, HCl, and ethanol), Kellers Etchant (comprising nitric acid, HCl, HF and water), Klemm's reagent (comprising sodium thiosulfate, potassium metabisulfite and water), Kroll's reagent (comprising nitric acid, HF and water), Nital (comprising ethanol and nitric acid), Marble's reagent (comprising CuSO₄, HCl, and water), Murakami's reagent (comprising K₃Fe(CN)₆, KOH and water), Oberhoffers reagent (comprising FeCl₃, SnCl₂, HCl, ethanol and water), Picral (comprising ethanol and picric acid), Ralphs etchant (comprising FeCl₃, CuCl₂, HCl, ethanol and water), Schantz etchant (comprising FeCl₃, H₂SO₄, HCl, nitric acid, acetic acid and water), V2A etchant (comprising HCl, HBr, nitric acid and water), Weck's etchant (comprising ammonium bifluoride, HCl and water), Winsteard's reagent (comprising picric acid, HCl, surfactant, and ethanol), and others.

In some embodiments, the etchant is an etching solution comprising HNO₃.

In some embodiments, the etchant is an etching solution comprising NaOH.

The etching conditions may be calibrated to determine different exposure durations, etching solution compositions, and varying concentrations of the solutions, allowing to achieve controllable porosity profiles with uniform pore sizes. High concentrations and long etching times typically result in larger pores but may also lead to non-uniform distributions of the pores on the metallic surface. Lower concentrations and shorter etching times result in smaller pores with better controlled pore distribution and pore density.

Table 1 below demonstrates general conditions for achieving nanopores of different pore sizes.

TABLE 1
etching conditions.

| | | Time of | | Nanopore |
|---|---|---|---|---|
| Etchant* | Concentration | exposure | Metal | average size |
| HNO₃ | 20-35% | 30 mins-72 | Al, Ti, Au | 10-50 nm |
| | | hrs | | |
| HNO₃ | 50-65% | 72-150 hrs | Au | 50-150 nm |
| NaOH | 3-10M | 24-72 hrs | Ti, Au, Al | 50-150 nm |
| Any | Dilute concentrations | Minutes to | All metals | 10-50 nm |
| etchant | | hours | | |
| Any | High concentrations | Minutes to | All metals | 50-150 nm |
| etchant | | hours | | |
| Any | Dilute concentrations | Hours to days | All metals | 100-200 nm or |
| etchant | | | | no nanopores |
| Ant etchant | High concentrations | Minutes | All metals | 50-100 nm |

*the etchant provided in a solution form. The etchant may be any such solution comprising the indicated material. “Any etchant” is an etchant as known in the art and selected herein. “Dilute concentration” is a concentration between 10-30% etchant. “High concentration” is a concentration between 30 and 80% etchant. “All metals” is a metal as selected herein.

The invention further provides a process for manufacturing an object of the invention, the process comprising etching a metallic surface of an object to produce therein a plurality of nanopores and contacting the etched surface with a medium comprising nanoparticles (or with a powder comprising or consisting the nanoparticles) to permit docking of at least a portion of the nanoparticles in the plurality of nanopores.

Further provided is a process for manufacturing an object of the invention, the process comprising:
contacting an object having a metallic surface with an etchant to form a plurality of nanopores on the surface;
contacting the etched surface (the surface having a plurality of nanopores) with a medium (solution, dispersion or emulsion) comprising nanoparticles or with a powder comprising or consisting the nanoparticles and allowing said nanoparticles to occupy at least a portion of the plurality of nanopores.

The portion of the nanopores occupied or loaded by the nanoparticles may be as discussed herein.

In some embodiments, the process further comprises forming a protective coating or film of a degradable or biodegradable material on the metallic surface having been treated with the nanoparticles.

In some embodiments, the metallic surface is a surface of a metallic object. In some embodiments, the metallic surface is a surface of a non-metallic object having been formed with a metallic external layer or surface.

As noted herein, the process comprises contacting the etched surface with a medium (solution, dispersion or emulsion) or a powder consisting or comprising nanoparticles and allowing said nanoparticles to occupy at least a portion of the plurality of nanopores. The contacting of the etched surface with the medium or the powder containing the nanoparticles may be generally described as mechanical deposition. In this deposition approach, the medium of nanoparticles may be simply positioned or deposited on the metallic surface. Alternatively, the metallic surface may be dipped, brushed, dusted or wetted with the medium. Where the nanoparticles are provided in a powder form, the powder may consist only the nanoparticles (single type or a population thereof), or a powder of the nanoparticles in a solid carrier. As the nanoparticles are designed and fabricated to fit the nanopore size, proper entrapment or docking in the nanopores is easily facilitated. The high surface area of porous metallic surface also improves the loading capacity of the metal to absorb the nanoparticles. The incorporation of nanoparticles does not affect the external appearance of the metallic surface which is a considerable factor when aesthetic parameters are accounted for.

The invention further provides a process for endowing a metallic surface with at least one property, the process comprising etching a metallic surface of an object to produce therein a plurality of nanopores and contacting the etched surface with a medium comprising nanoparticles to permit docking of at least a portion of the nanoparticles in the plurality of nanopores, wherein the nanoparticles are chemically or physically functional, thereby endowing the metallic surface with a chemical or physical functionality.

The chemically or physically functional nanoparticles may be selected amongst conductive nanoparticles, insulator nanoparticles, metallic nanoparticles, magnetic nanoparticles, catalyst nanoparticles, optically-active nanoparticles, hard or soft nanoparticles and others. Using such nanoparticles endows the metallic surface with one or more of conductive, insulator, catalytic, optical, hard or soft properties.

The invention further provides:

An object having a metallic surface with a chemically etched surface porosity comprising a plurality of etched surface nanopores, said plurality of nanopores being of a predefined porosity profile and containing nanoparticles of a material different from the metal forming the surface.

In some configurations of objects of the invention, the object is a metallic object or a non-metallic object having a metallic surface.

In some configurations of objects of the invention, the porosity profile comprises a pore size, pore density, pore distribution and/or pore concentration.

In some configurations of objects of the invention, the metallic surface is formed of a metal, a metal alloy, a metal oxide or a metal containing composite material.

In some configurations of objects of the invention, the metallic surface is or comprises a metal selected from aluminum, gold, silver, titanium, tungsten, copper, nickel, iron, chromium, and tin.

In some configurations of objects of the invention, the metallic surface is or comprises an aluminum alloy, a copper alloy, brass, bronze, cast iron, a nickel alloy, a nickel superalloy, stainless steel, high carbon steel or a low carbon steel.

In some configurations of objects of the invention, the nanopores having an average size between 10 and 500 nm.

In some configurations of objects of the invention, the nanopores average size is between 10 and 400, 10 and 300, 10 and 200, 10 and 100, 10 and 90, 10 and 80, 10 and 70, 10 and 60, 10 and 50, 10 and 40, 10 and 30, 10 and 20, 15 and 200, 15 and 150, 15 and 100, 15 and 95, 15 and 90, 30 and 150, 40 and 150, 50 and 150, 60 and 150, 70 and 150 or between 50 and 500 nm.

In some configurations of objects of the invention, the nanoparticles are provided solely in the chemically etched nanopores.

In some configurations of objects of the invention, the chemically etched surface comprising the nanopores containing nanoparticles is not provided with an external coating.

In some configurations of objects of the invention, the object is provided with a degradable or biodegradable external coating.

In some configurations of objects of the invention, the external coating is formed of a degradable or a biodegradable material selected amongst cellulose, starch, polyhydroxyalkanoates, polylactide, polycaprolactone, collagen, polyesters, polycarbonates, and polypeptides.

In some configurations of objects of the invention, the thickness of the coating is between 10 nm and 5 microns.

In some configurations of objects of the invention, the nanoparticles loaded into the nanopores have nanometric dimensions of a size capable of entering and docking within the nanopores and further selected from materials endowing the metallic object or surface with functional and/or mechanical properties.

In some configurations of objects of the invention, the nanoparticles are of a size that is between 1 and 10% smaller (in size or diameter) than the average size of the nanopores.

In some configurations of objects of the invention, the nanoparticles are selected from nanocarriers, nanospheres, nanocapsules, core/shell nanostructures, micelles, and matrix nanostructures formed of a matrix material.

In some configurations of objects of the invention, the nanoparticles are matrix nanoparticles formed of a polymer or a metal

In some configurations of objects of the invention, the nanoparticles are formed of a single material or are in a form capable of holding or containing a second or a further material.

In some configurations of objects of the invention, the nanoparticles are selected from a nanocarrier, a core/shell nanostructure or a matrix nanoparticle containing a material to be released.

In some configurations of objects of the invention, the nanoparticles are intended to modulate a mechanical, physical, chemical or a functional property of the metallic object or surface.

In some configurations of objects of the invention, the nanoparticles are selected amongst conductive nanoparticles, insulator nanoparticles, metallic nanoparticles, magnetic nanoparticles, catalyst nanoparticles, optically-active nanoparticles, hard nanoparticles and soft nanoparticles.

In some configurations of objects of the invention, the nanoparticles are selected from:
nanoparticles of a first material encapsulating or containing a second material;
nanoparticles of a material or a material composition having a predefined property; the nanoparticles being metallic nanoparticles, conductive nanoparticles, insulator nanoparticles, magnetic nanoparticles, catalyst nanoparticles, optically-active nanoparticles, hard or soft nanoparticles;
polymeric nanoparticles of a degradable or a biodegradable polymer;
polymeric nanoparticles of a degradable or biodegradable polymer containing at least one active material selected from drugs, cosmetic materials, fragrant materials, coloring materials, flavoring materials, antimicrobial materials, insecticidal materials, insect-repelling materials, agriculturally acceptable materials, wherein the at least one active material is released from the nanoparticles overtime;
non-polymeric nanoparticles of a degradable or biodegradable material containing at least one active material selected from drugs, cosmetic materials, fragrant materials, coloring materials, flavoring materials, antimicrobial materials, insecticidal materials, insect-repelling materials, agriculturally acceptable materials, wherein the at least one active material is released from the nanoparticles overtime;
polymeric nanoparticles of a degradable or biodegradable polymer containing at least one active material selected from drugs, cosmetic materials, fragrant materials, coloring materials, flavoring materials, antimicrobial materials, insecticidal materials, insect-repelling materials, agriculturally acceptable materials, wherein the at least one active material is to be released from the nanoparticles over time, and wherein the degradable or biodegradable polymer is a functional material;
non-polymeric nanoparticles of a degradable or biodegradable material containing at least one active material selected from drugs, cosmetic materials, fragrant materials, coloring materials, flavoring materials, antimicrobial materials, insecticidal materials, insect-repelling materials, agriculturally acceptable materials, wherein the at least one active material is to be released from the nanoparticles over time, and wherein the degradable or biodegradable material is a functional material.

In some configurations of objects of the invention, the nanoparticles are polymeric nanoparticles, optionally formed of a polymer selected from homopolymers, copolymers, terpolymer, block copolymers, and grafted polymers.

In some configurations of objects of the invention, the polymeric nanoparticles are formed of a polymer selected from polyolefins, poly acrylates and methacrylates, styrene polymers, polyesters, polyamides, polyimines, polycarbonates, natural polymers, cellulosic materials, polysaccharides, thermoplastic elastomers, polyvinyl alcohols, polynitriles, polyacetals, polyimides, polyarylketones, polyetherketones, polyhydroxyalkanoates, polycaprolactones, polyurethanes, polysulfones, polyphenylene oxides, polyphenylene sulfides, polyacetates, liquid crystal polymers, fluoropolymers, ionomeric polymers, thermoplastic elastomers, and blends thereof.

In some configurations of objects of the invention, the polymeric nanoparticles are formed of a polymer selected from gelatin, dextran, albumin, chitosan, alginate, polylactic acid (PLA), polyglycolic acid (PGA), copolymer of lactic acid and glycolic acid (PLGA), poly(ε-caprolactone) (PCL), polyalkylcyanoacrylate (PACA), poly(ethylene glycol) (PEG), poly (D,L-lactide-co-glycolide) (PLG), polyethyleneimine (PEI), poly (L-lysine), m-PEG-b-PLA.

In some configurations of objects of the invention, the nanoparticles are formed of a non-polymeric material selected from metallic or metal containing nanoparticles, wherein the metal may be different from the metal of the metallic object or surface.

In some configurations of objects of the invention, the metallic nanoparticles are selected amongst aluminum, gold, silver, titanium, tungsten, copper, nickel, iron, chromium, tin, or metal oxide or metal alloys thereof.

In some configurations of objects of the invention, the object is for use in medicine, cosmetics, electronics, optics, agriculture, chemical catalysis, food industry, diagnosis, or in thermal insulation.

In some configurations of objects of the invention, the object is for use in a method of delivery of at least one active material contained in said nanoparticles; or for inducing or modulating or increasing a mechanical property of the metallic object.

The invention also provides an object or an implant or an article having a metallic surface with a plurality of nanopores provided in a pore pattern formed by chemical etching of the metallic surface, the plurality of chemically etched nanopores comprising, holding, docking or hosting polymeric nanoparticles comprising at least one pharmaceutical, drug or active agent, wherein the polymeric nanoparticles are configured to degrade or biodegrade overtime to bring about release of the at least one pharmaceutical, drug or active agent.

In some configurations of objects of the invention, the at least one pharmaceutical, drug or active agent is a material used in medicine or veterinary.

In some configurations of objects of the invention is an implant or a medical device positioned against a tissue or an organ of a human or animal subject.

Also provided is an object or an article of manufacture having a metallic surface having a plurality of nanopores provided in a pore pattern formed by chemical etching of the metallic surface, the plurality of chemically etched nanopores comprising, holding, docking or hosting polymeric nanoparticles comprising at least one material, wherein the polymeric nanoparticles are configured to degrade or biodegrade overtime to bring about release of the at least one material.

In some configurations of objects of the invention, the at least one material contained in the nanoparticles is selected from agrochemicals, pharmaceuticals, cosmetic materials, personal care materials, laundering detergents, oral care materials, dental care materials, oil materials, water treatment materials, paint materials, flavoring materials, and fragrant materials.

In some configurations of objects of the invention, the at least one material is selected from pigments, dyes, colorants, scale inhibitors, emollient oils, insecticides, detergents, printing inks, corrosion and rust, inhibitors, antioxidants, catalysts, initiators, waxes, dispersants, flame retardants, biocides, anti-fouling agents, odor control agents, cosmetic additives, oxidizing agents, personal care actives, agrochemicals, fertilizers, fats, nutrients, enzymes, natural oils, fragrances, flavor and perfume oils, crop protection agents, medicaments, pharmaceuticals, and phase change materials.

Also provided is an object or an article of manufacture for use in agriculture, the object or article of manufacture having a metallic surface having a plurality of nanopores provided in a pore pattern formed by chemical etching of the metallic surface, the plurality of chemically etched nanopores comprising, holding, docking or hosting polymeric nanoparticles comprising at least one agriculturally acceptable material, wherein the polymeric nanoparticles are configured to degrade or biodegrade overtime to bring about release of the at least one agriculturally acceptable material.

In some configurations of objects of the invention, the at least one agriculturally acceptable material is an insecticidal material, an insect-repelling material, antibacterial material, antiviral material, antifungal material, nutrient, or a fertilizer.

Also provided is a process for manufacturing an object according to the invention, the process comprising contacting a metallic surface of an object having a plurality of surface nanopores with a medium comprising nanoparticles and allowing said nanoparticles to occupy at least a portion of the plurality of nanopores, wherein the surface nanopores have been formed by chemical etching.

In some configurations of processes of the invention, the process comprises treating the object to remove nanoparticles present outside of the nanopores.

In some configurations of processes of the invention, the process comprising obtaining the object having an etched metallic surface decorated with a plurality of nanopores.

In some configurations of processes of the invention, the process comprising etching a metallic surface of an object under conditions selected to form a plurality of surface nanopores having a predefined porosity profile.

In some configurations of processes of the invention, the etchant is selected from Adler etchant (comprising copper ammonium chloride, HCl, FeCl₃, water), ammonium persulfate etchant (comprising ammonium persulfate, and water), aluminum Al—NaOH etchant (comprising NaOH and water), etchant commercially available under ASTM No. 30 (comprising ammonia, H₂O₂, and water), etchant commercially available under ASTM No. 97 (comprising KOH and water), etchant commercially available under ASTM No. 157 (comprising CrO₃, HCL and water), Berahas etchant (comprising Na₂S₂O₃, K₂S₂O₅ and water), Carpenters stainless steel etchant (comprising FeCl₃, CuCl₂, HCl, nitric acid, and ethanol), etchant commercially available under Copper No. 1 (comprising nitric acid and water), etchant commercially available under Copper No. 2 (comprising HCl, ferric chloride and water), dichromate etchant (comprising K₂Cr₂O₇, H₂SO₄, NaCl and water), Frys reagent (comprising HCl, copper chloride, water and ethanol), Inconel etchant (comprising nitric acid, HCl, H₂O₂ and water), Kalling's No. 2 (comprising CuCl₂, HCl, and ethanol), waterless Kalling's reagent (comprising CuCl₂, HCl, and ethanol), Kellers Etchant (comprising nitric acid, HCl, HF and water), Klemm's reagent (comprising sodium thiosulfate, potassium metabisulfite and water), Kroll's reagent (comprising nitric acid, HF and water), Nital (comprising ethanol and nitric acid), Marble's reagent (comprising CuSO₄, HCl, and water), Murakami's reagent (comprising K₃Fe(CN)₆, KOH and water), Oberhoffers reagent (comprising FeCl₃, SnCl₂, HCl, ethanol and water), Picral (comprising ethanol and picric acid), Ralphs etchant (comprising FeCl₃, CuCl₂, HCl, ethanol and water), Schantz etchant (comprising FeCl₃, H₂SO₄, HCl, nitric acid, acetic acid and water), V2A etchant (comprising HCl, HBr, nitric acid and water), Weck's etchant (comprising ammonium bifluoride, HCl and water), Winsteard's reagent (comprising picric acid, HCl, surfactant, and ethanol).

In some configurations of processes of the invention, the etchant is an etching solution comprising HNO₃.

In some configurations of processes of the invention, the etchant is an etching solution comprising NaOH.

In some configurations of processes of the invention, the process comprising etching a metallic surface of an object to produce therein a plurality of nanopores and contacting the etched surface with a medium comprising nanoparticles or with a powder comprising or consisting the nanoparticles to permit docking of at least a portion of the nanoparticles in the plurality of nanopores.

Also provided is a process for manufacturing an object according to claim 1, the process comprising:
contacting an object having a metallic surface with an etchant to form a plurality of nanopores on the surface;
contacting the etched surface with a medium (solution, dispersion or emulsion) comprising nanoparticles or with a powder comprising or consisting the nanoparticles and allowing said nanoparticles to occupy at least a portion of the plurality of nanopores.

In some configurations of processes of the invention, the process further comprises forming a protective coating or film of a degradable or biodegradable material on the metallic surface having been treated with the nanoparticles.

Also provided is a process for endowing a metallic surface with at least one property, the process comprising etching a metallic surface of an object to produce therein a plurality of nanopores and contacting the etched surface with a medium comprising nanoparticles to permit docking of at least a portion of the nanoparticles in the plurality of nanopores, wherein the nanoparticles are chemically or physically functional, thereby endowing the metallic surface with a chemical or physical functionality.

In some configurations of processes of the invention, the chemically or physically functional nanoparticles are selected amongst conductive nanoparticles, insulator nanoparticles, metallic nanoparticles, magnetic nanoparticles, catalyst nanoparticles, optically-active nanoparticles, hard or soft nanoparticles.

In some configurations of processes of the invention, the process is for endowing the metallic surface with one or more of conductive, insulator, catalytic, optical, hard or soft properties.

The invention further provides a kit comprising at least one etchant or an etching solution, nanoparticles and instructions of use, wherein the etchant or etching solution is selected to generate nanopores in an object having a metallic surface and wherein the nanoparticles are selected to be loaded into said nanopores.

In some embodiments, the instructions direct a user to generating the nanopores in an object of their choosing.

In some embodiments, the kit may further comprise a medium for solubilizing the etchant.

In some embodiments, the nanoparticles may be provided in a liquid medium (as a solution, a suspension, an emulsion or a dispersion), or provided as a powder comprising or consisting the nanoparticles.

In some embodiments, the kit may further comprise means for applying or depositing the etchant or etching solution on the metallic surface of the object.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1. Thymol HPLC calibration. HPLC calibration curve using thymol samples from 4 μg/ml to 180 μg/ml (calibration linear factor R²=0.9972).

FIG. 2. SEM characterization of the different metal surfaces. These images represent the surface structure of the different metals before etching treatments: aluminum, 9-karat gold alloy and titanium.

FIGS. 3A-B. EDS characterization of the different metal surfaces. (A) EDS measurements showing the chemical element contents were realized for each metal sample: aluminum, 9-karat gold alloy, and titanium. Y-axis depicts the number of counts and the x-axis the energy of the X-rays. The position of the peaks leads to the identification of the elements and the peak height helps in the quantification of each element's concentration in the sample. The aluminum sample contains 97.9% Al; the 9-karat gold alloy sample contains: 35.% Cu, 8.9% Zn, 37.8% Au, and 10.3% Ag; and the titanium sample contains 99.2% Ti. (B) EDS measurement was directly realized on SEM image showing the different surface zones containing different chemical elements besides Au.

FIGS. 4A-B. SEM characterization of the different porous metal surfaces. These images represent the nanostructures obtained in the optimal conditions of fabrication. (A) Aluminum nanostructures with about 30 nm pore size were obtained after dipping in HNO3 35% for 35 min, gold nanostructures with about 30 nm pore size were obtained after dipping in HNO3 35% for 72 h and titanium nanostructures with about 150 nm pore size were obtained after dipping titanium in NaOH 5M for 24 h. (B) This picture represents the different metal pieces before and after the etching treatment: titanium (1. before, 2. after), 9-karat gold alloy (3. before, 4. after), aluminum (5. before, 6. after).

FIGS. 5A-B. Physicochemical characteristics of the different metal surfaces. (A) Vickers hardness measurements performed on titanium, 9-karat gold alloy and aluminum before etching treatment [A], 24 h after [B] and 3 months after [C]. (B) Water contact angle measurements on titanium, 9-karat gold alloy and aluminum before etching treatment [A] and 24 h after [B]. Representative Images from each experiment is presented below the graphs. ns indicates no significant difference, n=3, * p<0.1, *** p<0.001

FIGS. 6A-E. Thymol-loaded PLGA nanoparticle preparation and characterization. (A) Illustration of the encapsulation process of thymol into PLGA nanoparticles by emulsification-evaporation method (reproduced and adapted with authorization. (B) DLS measurement of the nanoparticles (C) Zeta potential of the nanoparticles (D) SEM and (E) TEM Image of the nanoparticles.

FIGS. 7A-E. Thymol-loaded PLGA nanoparticles encapsulation in porous titanium structure. (A) SEM image of thymol-loaded PLGA nanoparticles in porous titanium (PLGA particles were post-highlighted in yellow). (B) Thymol release profile measurement using HPLC (from PLGA nanoparticles vs. free thymol). * p<0.05, ** p<0.001. (C) Thymol release profile measurement using odor test (from PLGA nanoparticles vs. free thymol). * p<0.05, ** p<0.001. (D) 3D printed titanium ring prototype before (left) and after etching treatment (right). (E) SEM image of the 3D printed titanium ring after etching treatment.

FIGS. 8A-B. SEM characterization of nanoparticles entrapped in aluminum and gold porous metal surfaces. (A) mPEG-b-PLA nanoparticles entrapped in porous aluminum. (B) Silica nanoparticles entrapped in porous gold.

FIG. 9. TEM image of a titanium stent surface according to the invention.

FIGS. 10A-C. Nanoparticles characterization by DLS. (A) PLGA (50:50) nanoparticles for the hydrophobic drug Paclitaxel, showing a particles size ~170 nm. (B) PLA nanoparticles for the hydrophilic drug Dexamethasone, showing a particles size ~100 nm. (C) PLA nanoparticles for the hydrophilic drug Dexamethasone, showing a Zeta potential −15.53 mV.

FIGS. 11A-B. Loading nanoparticles into porous titanium. PLGA NPs Loaded into porous titanium with 2 different concentrations: (A) 0.5 ug/mL, and (B) 0.1 ug/mL.

FIGS. 12A-C. PLA nanoparticles loaded into porous titanium with 3 different concentrations: (A) 0.5 ug/mL, (B) 0.1 ug/mL, and (C) 0.05 ug/mL.

### DETAILED DESCRIPTION OF EMBODIMENTS

### Materials and Methods

### Metal Sample Preparation

Pure titanium and aluminum foils (99% purity, 25×25×0.25 mm³) were purchased from Alfa Aesar (MA, USA), and 9-karat yellow gold sheets (10×7×0.2 mm³) were purchased from Rashbel (Israel). To produce nanoporous structures, the titanium samples were dipped in 5, 7.5, or 10 M NaOH (Bio-Lab Ltd, Israel) aqueous solutions for 24 h or 48 h; gold and aluminum samples were dipped in 15% or 35% HNO₃ (Acros Organics, Belgium) aqueous solutions for different durations. The samples were washed with DDW for 1 min and dried under a nitrogen stream.

### Thymol-Loaded PLGA Particle Preparation and Deposition

Nanoparticles were prepared using an emulsification-evaporation method. The oily phase was formed by dissolving 100 mg of PLGA 50:50 lactic according to a glycolic acid ratio (Sigma-Aldrich, MA, USA) and 20 mg of thymol (MW=150.22 g/mol; ThermoFisher, MA, USA) in 10 ml of acetonitrile (Bio-Lab Ltd., Israel) containing 0.01% Tween 80 (ThermoFisher, MA, USA). While being stirred, the aqueous phase of Solutol 0.1% (Glentham Life Sciences, UK) was added to the oily phase and stirred for 10 min using an overhead stirrer at 550 rpm. Next, the solution was transferred into a round bottom flask and connected to a rotary evaporator (Heidolph, Germany). After the solvent was entirely evaporated, the nanoparticles were centrifuged at 10000 rpm for 5 min to remove impurities. A solution of 0.5 mg/mL thymol-loaded PLGA particles was deposited on porous titanium. mPEG-b-PLA micelles, prepared by a dialysis method as previously described, with a size of 30 nm, and silica nanoparticles, purchased from Nanocomposix (CA, USA), with a size of 50 nm, were deposited on porous aluminum and gold surfaces. The volume of suspended nanoparticles was 20 μl for a 1 cm by 1 cm piece of metal; evaporation was carried out overnight at room temperature.

### Physical and Chemical Characterization of Metal Substrates and Nanoparticles

The nanoparticles' mean size, range, and charge were measured using dynamic light scattering and Zetasizer (Zetasizer Nano ZSP, Malvern Instruments) at 25° C. For scanning electron microscopy (SEM, Magellan 400 L ThermoFisher, a former FEI Company, USA), the metal and particle samples were sputtered with a thin iridium layer. For transmission electron microscopy (TEM, JEM-1400 Plus, JEOL, Japan), the particle samples were initially stained with 2.5% uranyl acetate, and 5 μL of the particle suspension was placed on formvar/carbon-coated copper 200 mesh grids and mixed with 5 μL of NanoVan (Nanoprobes, NY, USA) for 10 s. After the extra stain agent was removed, the grids were then dried and measured. A high-resolution SEM (Apreo 2S, ThermoFisher, a former FEI Company, USA) equipped with energy-dispersive X-ray spectroscopy (EDS) technology was utilized to analyze the samples' chemical elemental composition.

Microhardness indentation measurements were performed to assess the mechanical stability of the samples. Vickers hardness was measured using a 100-gf load and a 15-s loading time (Duramin-40, Struers, Denmark) on each metal before etching treatment, 24 h after, and 3 months after. To evaluate the metal surface energy, contact angles for 5 μL of DDW droplets were measured on each surface. The contact angles were measured after processing by ImageJ free software using a contact angle plugin. For these measurements, aluminum samples were dipped in HNO₃ 35% for 35 min, gold samples were dipped in HNO₃ 35% for 72 h and titanium samples were obtained were dipped in NaOH 5M for 24 h after which they were washed with DDW for 1 min and dried under a nitrogen stream.

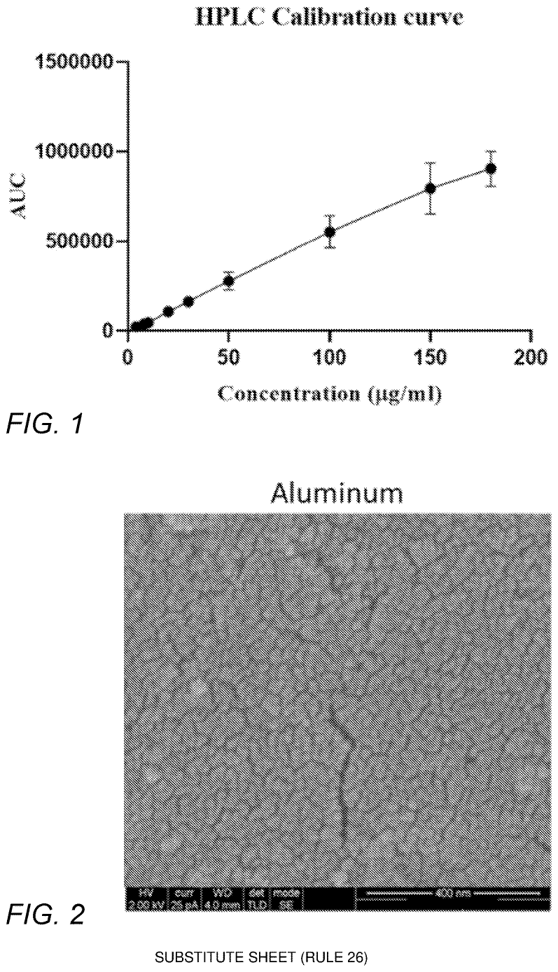

To determine the amount of thymol, we used high-performance liquid chromatography (HPLC) analysis. Chromatographic conditions were obtained using a Shimadzu model LC-20 instrument with a photodiode array (PDA) detector (Shimadzu, Japan). Acetonitrile and water (HPLC grade, Baker, ME, USA) were utilized as the organic mobile phase in a 50:50 ratio. The flow rate was 1 ml/min with an injection sample volume of 10 μL into a C18 column (Gemini-NX 5u, C18 110A, 250×4.6 mm, Phenomenex, CA, USA). The temperature was set at 20° C., and the detection was monitored at a 275 nm wavelength. The unique peak of thymol was detected after 2.7 min. Thymol standard solutions in acetonitrile were prepared and used for calibration (FIG. 1).

### Thymol Retention Profiles on Metals and a Smell Test

Thymol-loaded PLGA samples were prepared in advance and dried in a hood. Samples containing free thymol in acetonitrile were also prepared and dried in the hood to establish a reference control. The samples were left in the open air at room temperature and were provided by 5 random volunteers (20 to 30 years old) who smelled the sample every day and provided a score for odor intensity ranging from 0 to 10. For quantitative analysis of the thymol release, the samples were dissolved in acetonitrile at different time points to determine the remaining amount of thymol using HPLC analysis using the same conditions as for content analysis of nanoparticles.

### Titanium 3D Printing

Jewelry titanium objects were designed with AutoCAD® (version 2018.3, Autodesk, Inc., San Rafael, CA, USA), saved in their final form in STL format, and produced using an online 3D printing platform (i.materialise, Materialise, Belgium). The objects were printed using a titanium alloy called Ti—Al6-V4 by Select Laser Melting (SLM) technology. A high-powered laser selectively binds particles on the powder bed together while the machine distributes even layers of metallic powder. Support structures are automatically generated, built simultaneously in the same material, and later manually removed. Once complete, the part underwent heat treatment.

### Results

### Aluminum Porous Structures

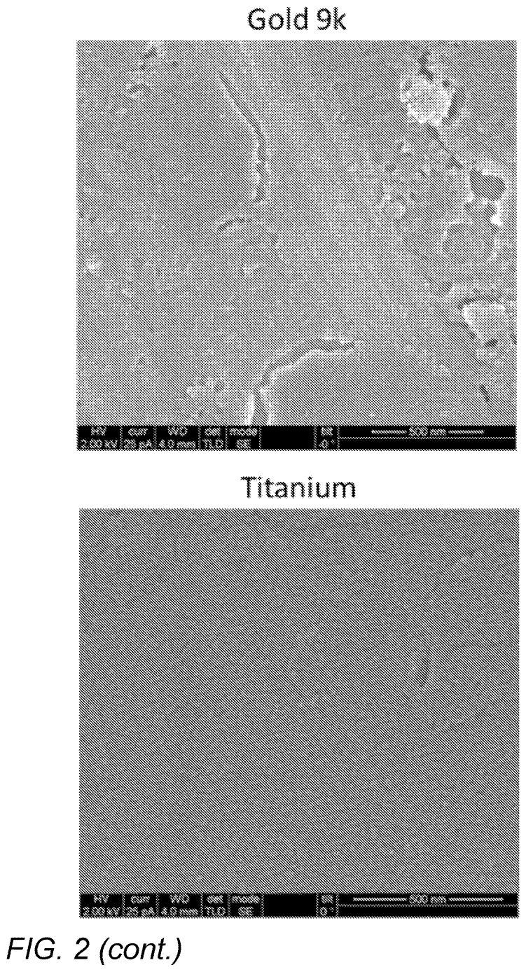

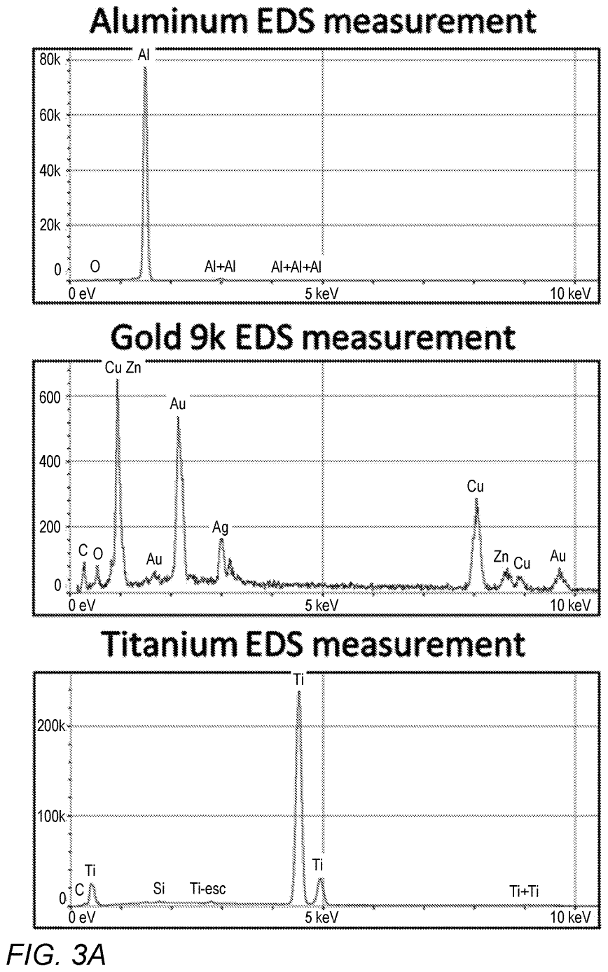

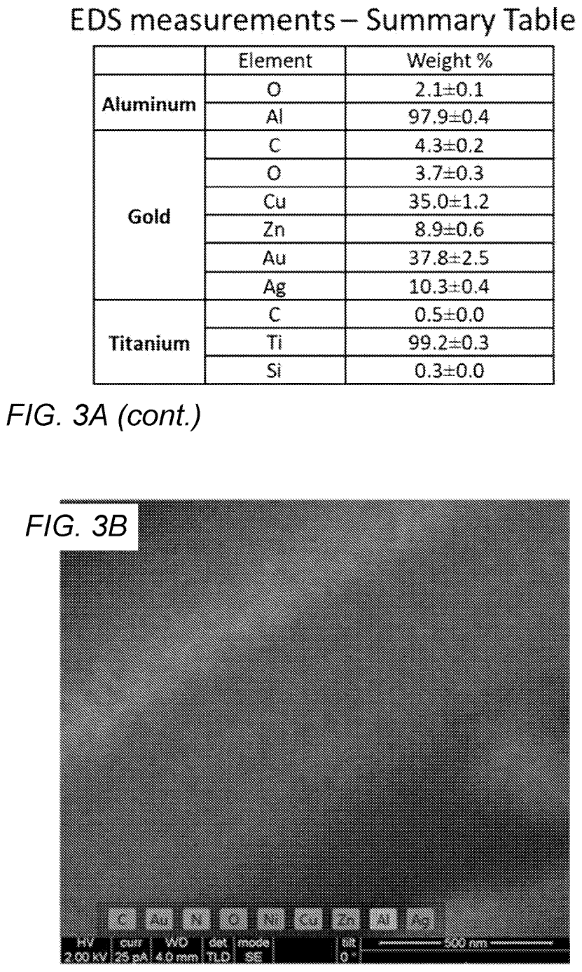

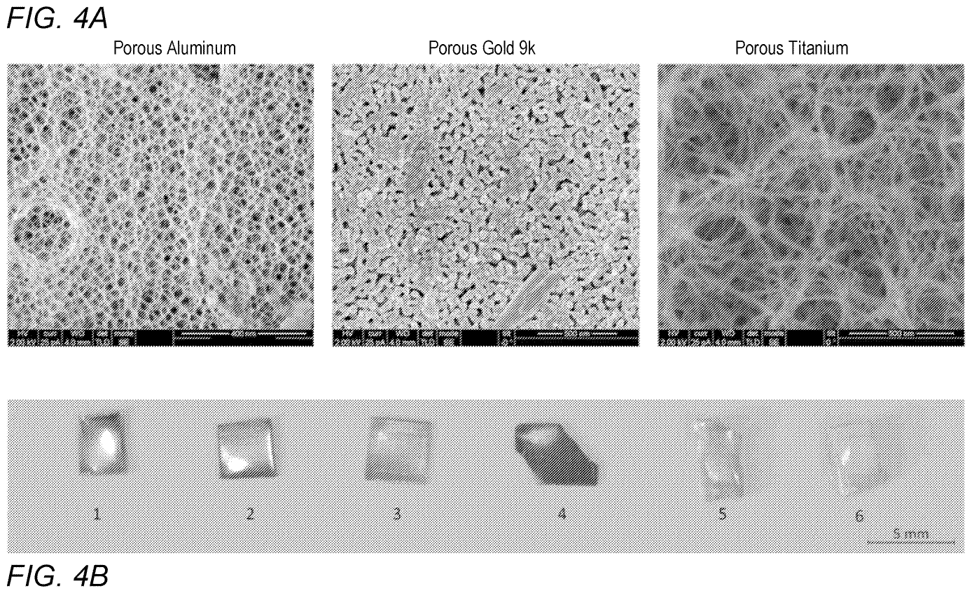

Aluminum reacts with nitric acid (HNO₃) to form porous structures on the surface of the metal. Aluminum samples have a non-porous structure, as shown in SEM before HNO₃ treatment (FIG. 2), as confirmed by EDS analysis, showing that the samples were mainly composed of Al elements (FIG. 3A). Aluminum pieces were dipped in various HNO₃ concentrations at different times (Table 1) to determine the ideal conditions to obtain porous structures. Only a few porous structures were observed at short times and low concentrations (from 15 min at 15% and 30% to 30 min at 15%). However, the etching treatment was aggressive at longer times of 1, 4, and 24 h and at higher concentrations of 53% and 70%, destroying the porous structures. Only at the intermediate concentration of 35% during 30 min to 1 h of exposure were stable porous structures with a 30 nm diameter produced (FIG. 4).

### Gold Porous Structures

The gold alloy we used also has a smooth and non-porous structure, based on SEM measurements (FIG. 2). According to the EDS analysis, the gold alloy only comprises 37.8% Au; the remaining elements are 35% Cu, 8.9% Zn, and 10.3% Ag (FIG. 3A). FIG. 3B presents the distribution of the chemical elements on the gold alloy surface before HNO₃ etching. The non-gold chemical components were chemically etched away by reacting them with HNO₃, leaving only pure gold. (FIG. 4). The optimal conditions for obtaining large nanopores were determined using different concentrations and soaking durations. At a short soaking time of 24 h, only a high concentration of 35% provided pores with a size of about 30 nm, whereas a lower concentration of 15% induced smaller (15 nm) or only sporadic pores. At longer soaking times of 72 h, 30 nm pores were obtained at both concentrations (15% and 35%). Longer soaking times of 96 to 146 h in HNO₃ were too aggressive and not suitable for obtaining uniform pores (Table 2).

TABLE 2
Porous metals prospecting.

| | Solution | Time (h) | Concentration | Results |
|---|---|---|---|---|
| Aluminium | HNO₃ | 0.25 | 15% | few pores |
| | | | 35% | few pores |
| | | 0.5 | 15% | few pores |
| | | | 35% | ~30 nm pores |
| | | | 53% | no pores - aggressive |
| | | | | treatment |
| | | | 70% | no pores - agressive |
| | | | | treatment |
| | | 1 | 15% | few pores |
| | | | 35% | ~30 nm pores |
| | | 4 | 15% | no pores - aggressive |
| | | | | treatment |
| | | | 35% | no pores - aggressive |
| | | | | treatment |
| | | 24 | 15% | no pores - aggressive |
| | | | | treatment |
| | | | 35% | no pores - aggressive |
| | | | | treatment |
| Titanium | NaOH | 24 | 5M | ~150 nm pores |
| | | | 7.5M | no pores - aggressive |
| | | | | treatment |
| | | | 10M | no pores - aggressive |
| | | | | treatment |
| | | 48 | 5M | ~150 nm pores |
| | | | 7.5M | no pores - aggressive |
| | | | | treatment |
| | | | 10M | no pores - aggressive |
| | | | | treatment |
| Gold 9k | HNO₃ | 0.5 | 35% | no pores - weak treatment |
| | | 24 | 15% | no pores - weak treatment |
| | | 24 | 35% | ~30 nm pores |
| | | 48 | 15% | ~15 nm pores |
| | | 48 | 35% | ~30 nm pores |
| | | 72 | 15% | ~30 nm pores |
| | | 72 | 35% | ~30 nm pores |
| | | 96 | 35% | ~15 nm - aggressive |
| | | | | treatment |
| | | 121 | 35% | ~15 nm - aggressive |
| | | | | treatment |
| | | 146 | 35% | ~15 nm - aggressive |
| | | | | treatment |

### Titanium Porous Structures

A series of different sodium hydroxide (NaOH) soaking solutions were prepared to determine the optimal conditions to produce titanium with large pores. SEM images of the titanium sample before chemical treatment showed a smooth structure (FIG. 2), and EDS measurements confirmed a content of 99.2% Ti elements (FIG. 3A). Only by using a 5 M NaOH solution for 24 h, was it possible to obtain porous structures ranging between 150 and 200 nm in diameter (FIG. 4). High concentrations of NaOH (7.5 and 10 M) were too aggressive and did not induce controllable porous structures. However, extending the duration of chemical treatments to 48 h had no impact on the enlargement of the porous structures (Table 2).

### Physicochemical Characteristics of the Metal Surface

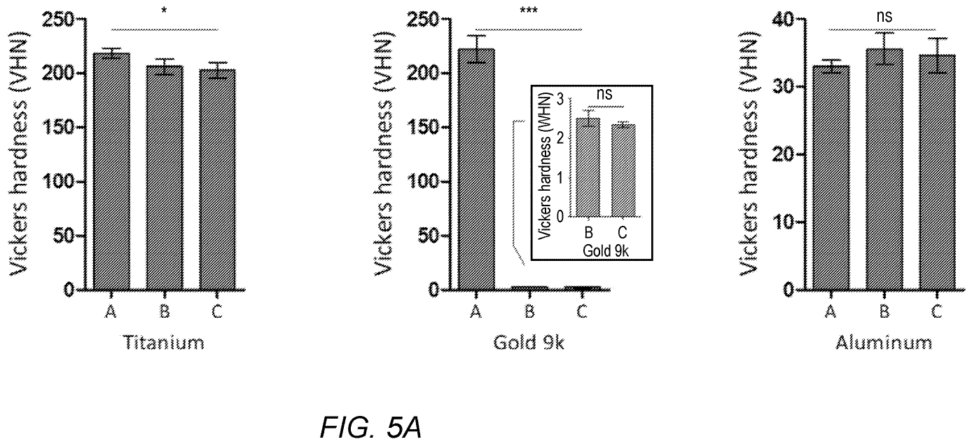

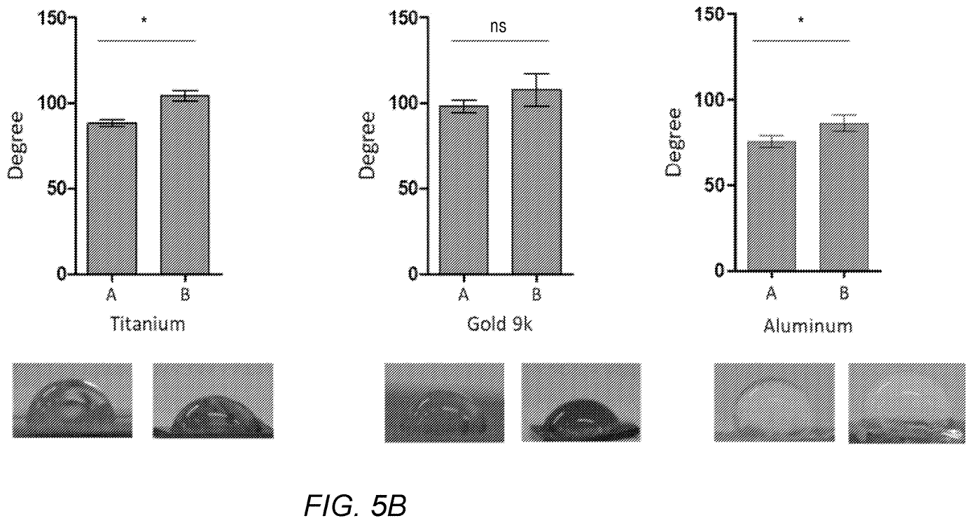

To understand the long-term performance of the materials and the impact of the process on the samples, Vickers hardness measurements were performed before and after the etching treatment and 3 months post treatment. In the titanium surface, a slight decrease in the hardness properties was observed post etching, that remained unchanged over 3 months. In aluminum samples a neglectable mechanical increase was observed post etching. Nevertheless, a significant decrease in the hardness of the gold samples after the dealloying etching process was measured (FIG. 5A). Contact water angle measurements showed that the etching process improved the wettability of the samples (FIG. 5B). This result is correlated with the formation of pores on the surface of these metals.

### Thymol Encapsulation in PLGA Nanoparticles

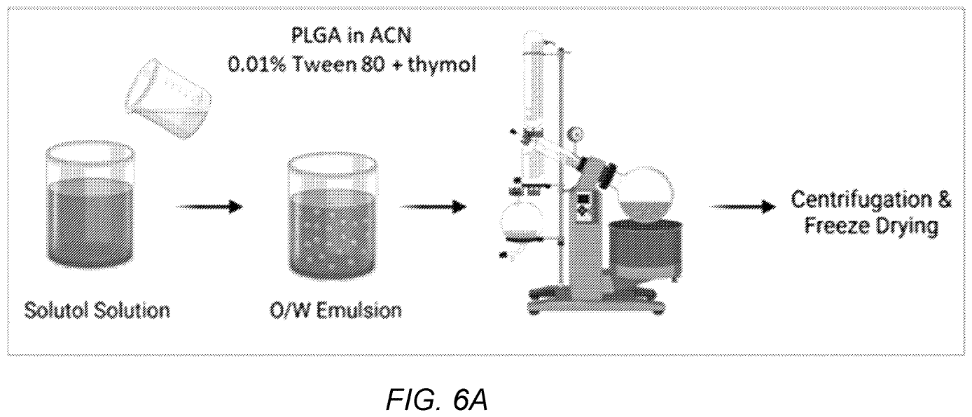

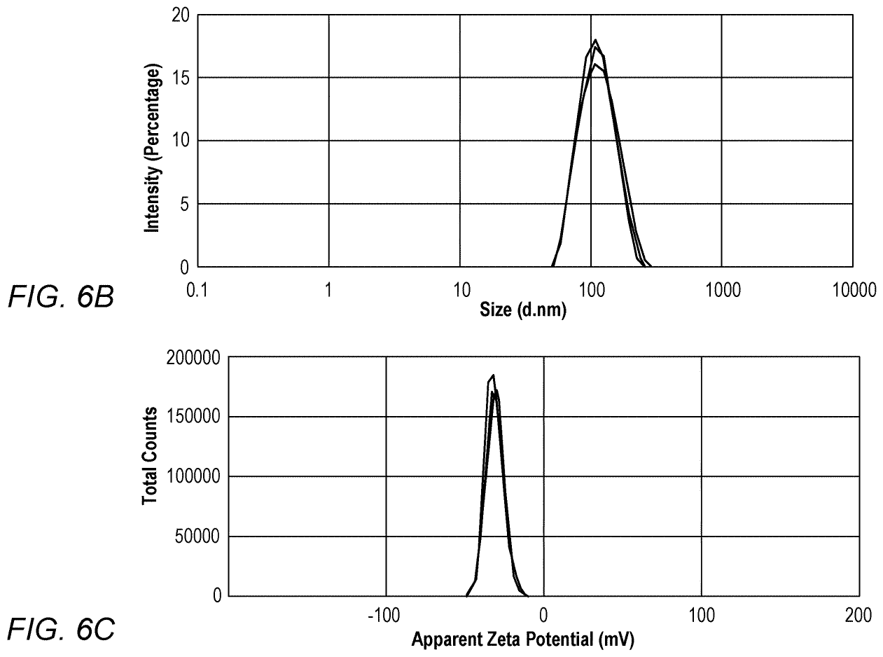

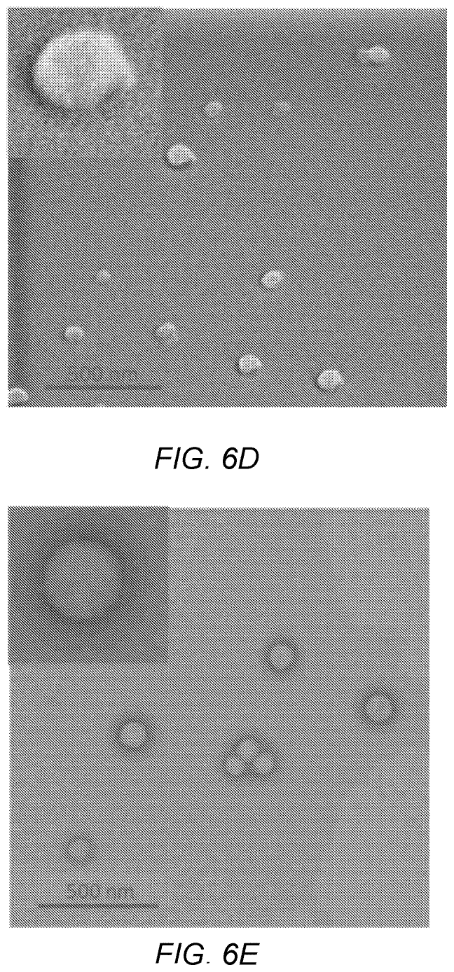

Thymol was encapsulated in PLGA nanoparticles using an emulsion solvent evaporation technique, shown in FIG. 6A. Thymol is a small hydrophobic molecule that can be entrapped in the matrix material during particle emulsification. The nanoparticle's size was measured using the DLS method (165.6 nm+/−2.227; PDI=0.079+/−0.018) (FIG. 6B). The nanoparticles were negatively charged (−32.1 mV+/−0.681) (FIG. 6C); the morphology of the particles was spherical with high uniformity, which can be shown by SEM and TEM microscopy (FIGS. 6D-E).

The Release of Thymol-Loaded Nanoparticles from the Porous Material

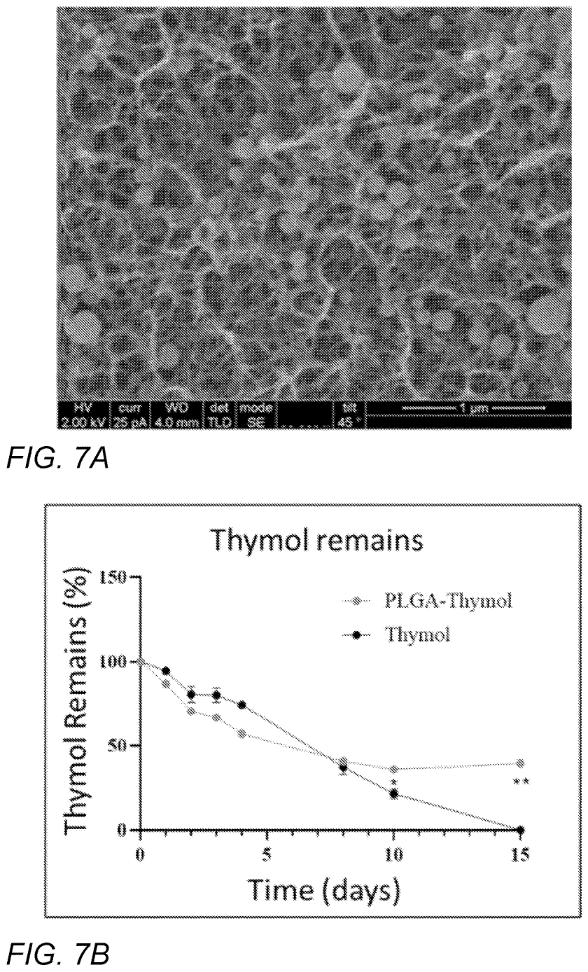

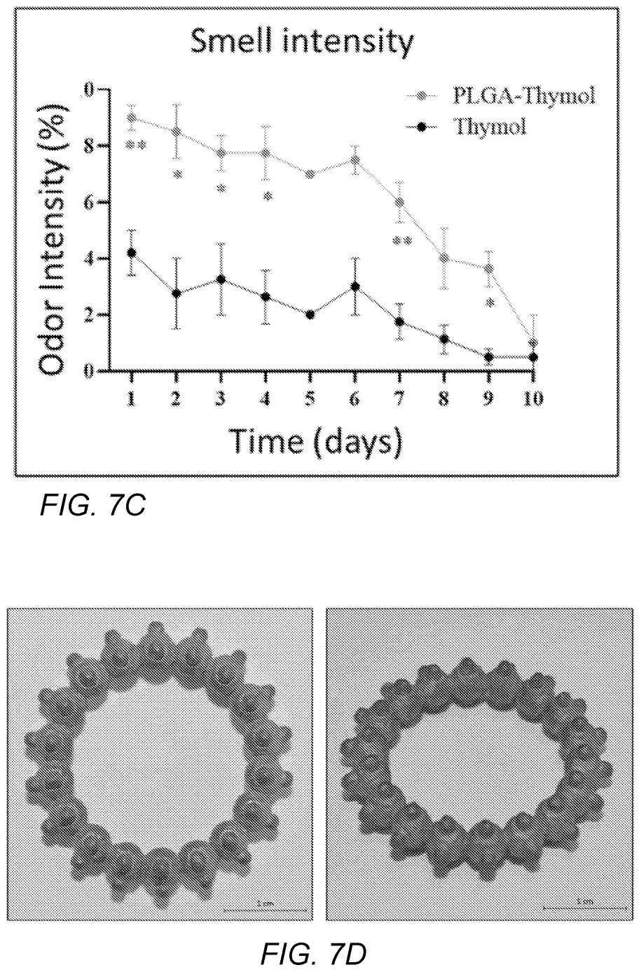

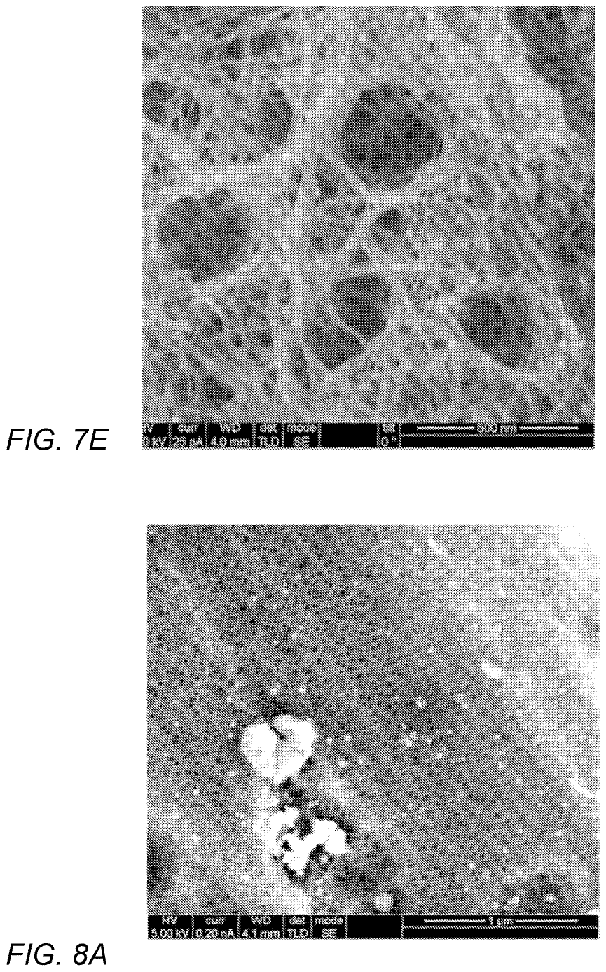

The release profiles of thymol from the nanoparticle-loaded metals were compared with those of free thymol in open-air conditions. The nanoparticles were efficiently internalized into the porous titanium, with minimal residues on the outer surface, according to the SEM images (FIG. 7A). The release profile confirmed that after 15 days, the amount of thymol remaining in the samples was significantly higher than that of the free thymol. After 15 days, there was almost no thymol left in the free thymol samples, and there was nearly 40% of thymol when it was used in particles (FIG. 7B). Odor tests of the same samples for 10 days revealed that the samples containing thymol-loaded PLGA nanoparticles obtained higher scores for smell intensity than those that contained thymol, despite the identical initial quantities (FIG. 7C). Based on SEM, mPEG-b-PLA micelles (30 nm) were also deposited on porous aluminum, and silica nanoparticles (50 nm) were deposited on porous gold, confirming efficient incorporation into the metals (FIG. 8).

To provide a full prototype for smell-releasing jewelry, a titanium ring was designed and 3D printed. FIG. 7D shows pictures of the titanium 3D-printed ring before and after treatments, showing no visual effects on the actual product. SEM images of the porous titanium printed ring were similar to the porous pure titanium structure (FIG. 7E).

### API Loaded in Porous Titanium Stent

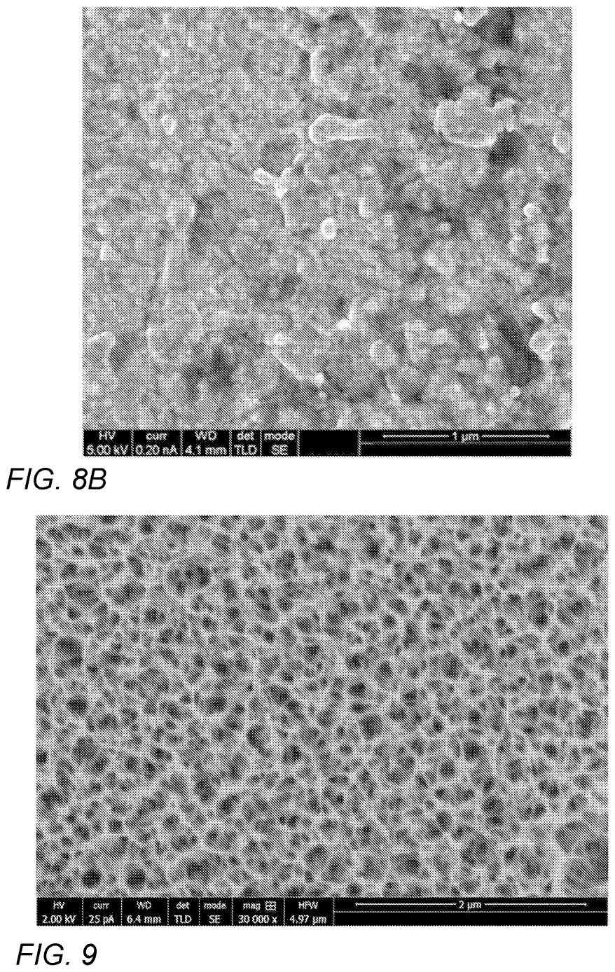

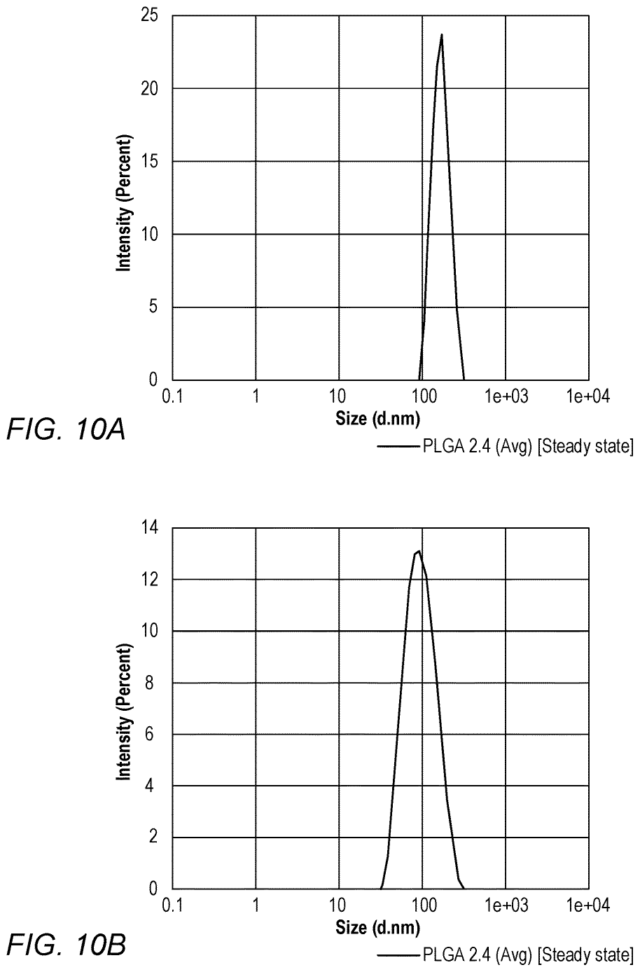

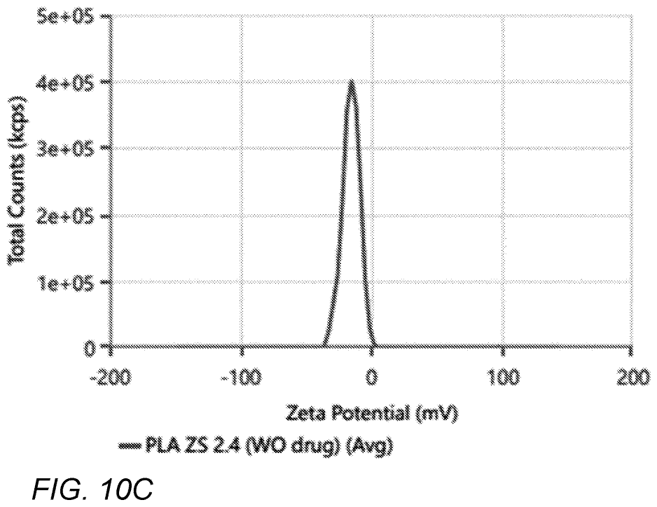

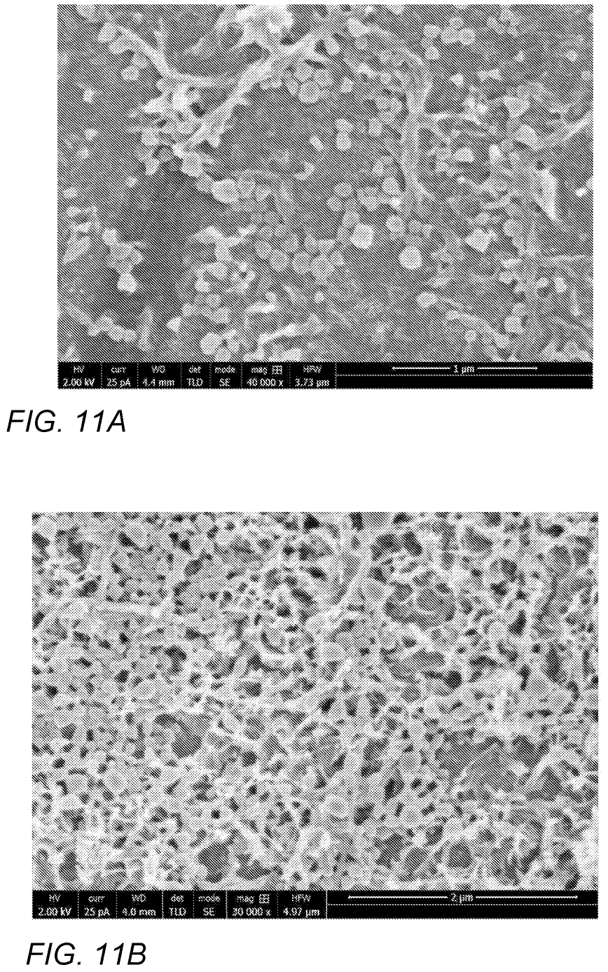

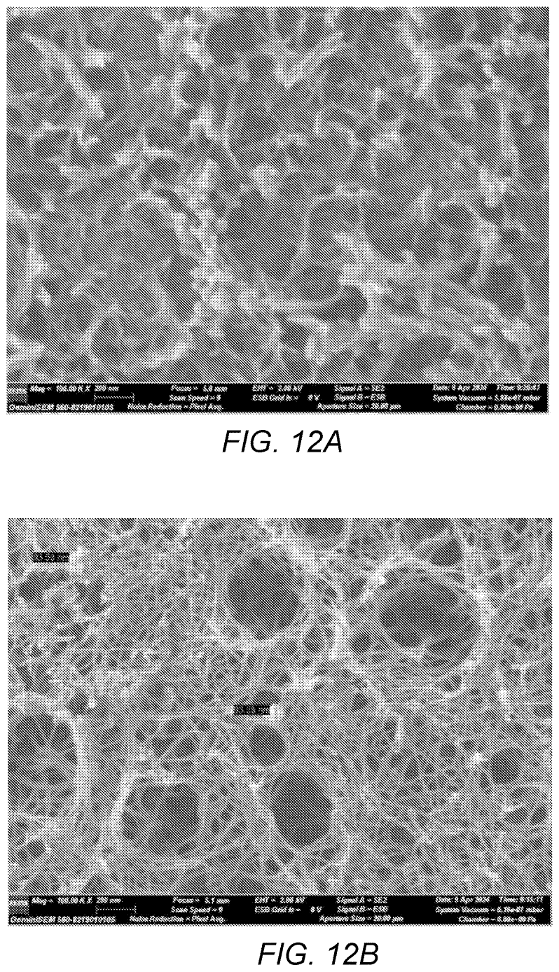

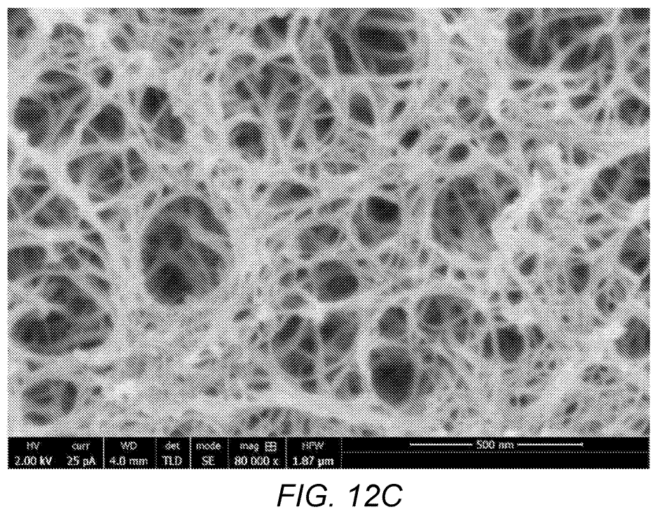

To demonstrate encapsulation of active pharmaceutical ingredients in a metal medical device, a stent was etched to provide surface nanopores. Various nanoparticles comprising paclitaxel or other drugs were formed and entrapped in the stent nanopores. FIGS. 9, 10, 11 and 12 demonstrate successful entrapment of the API loaded nanoparticles in the metal stent. In short, PLGA loaded particle, either O/W with paclitaxel or W/O/W with dexamethasone were made. The solution of the particles was placed on the metal surface (pretreated with PDDA to enhance particle adhesion) and dried in hood. The metal/particle samples were dipped in PBS and samples were collected at various time points to measure drug release by HPLC.

### DISCUSSION

In this study, we described a versatile and cost-effective method for fabricating porous metals with controllable pore sizes. Metal dipping in etching solutions is a simple and inexpensive alternative process, thus holding significant promise for many applications for porous metals. The etching conditions were extensively calibrated, including testing different exposure durations, etching solution compositions, and varying concentrations of the solutions, leading to metals with controllable and relatively uniform pores. We found that porous morphology is dependent on the type of solvent and that the pore size and distribution are impacted by the etching solvent concentrations and the etching durations. High concentrations and long etching times typically result in larger pores, but they may also lead to non-uniform distributions or even complete dissolution of the metal as in the case of aluminum using a HNO₃ concentration of 70% during 30 min or even using a HNO₃ concentration of 15% during 4 h or 24 h. At the same time, lower concentrations and shorter etching times result in smaller pores or even the absence of enough etching influence as can be shown in the case of aluminum using a HNO₃ concentration of 15% during 30 min or with 35% during 15 min. In the other materials similar adjustments made it possible to control the pore size of the final structure. According to our needs, we successfully created surfaces with pores of dozens of nanometers, which could be used for particle entrapment. In the future, some improvements, based on combining several solvents and integrating computational studies could be made to gain even finer control or to obtain larger pores to allow the incorporation of micron-sized particles using this method.

To provide a wider demonstration, we used different metals: aluminum, gold, and titanium as substrates. Etching processes in aluminum were previously described for different acid solvents; however, obtaining nanoporous structures remains a challenge, since most large micropores were obtained using hydrochloric acid (HCl), and an oxalic acid/phosphoric acid mixture (C₂H₂O₄/H₃PO₄). On the other hand, nanopores were obtained using an anodizing process involving sulfuric acid (H₂SO₄) dipping; however, the small dimensions of the pores prevented the further deposition of nanoparticles. In our case, when we used HNO₃ (35% during 35 min) as the etching chemical to obtain nanopores via a simple dipping process, a homogeneous structure with pore sizes of ~30 nm was formed enabling the deposition of mPEG-b-PLA micelles.

With gold surfaces, we used HNO₃ because of its common use as a method for determining the purity (karat) of gold by breaking down other surrounding chemical elements. This dealloying method is based on the observation that HNO₃ does not affect the gold elements. To date, it has been shown that this approach could produce tiny pores in a gold alloy, which range in size from 2 to 5 nm. Therefore, to create bigger pores, we used a 9-karat gold alloy with high quantities of non-gold chemical elements to take advantage of this principle and to produce rather wide pores of about 30 nm (using HNO₃ 35% for 72 h) enabling the deposition of silica nanoparticles. Further reducing the alloy purity could enhance the pore mesh size.

Titanium, unlike gold and aluminum, does not react with acid; therefore, NaOH is typically used to induce porosity. Our interest in titanium is beyond the scope of this study since this material is widely used in implantable devices and thus potentially benefits from our approach. Different NaOH concentrations and dipping conditions were used to optimize the production of uniformly porous titanium with controllable pore size and larger pores, which were ideal for incorporating slow-releasing particles. Finally, the ideal pore size was obtained by dipping the titanium samples in NaOH 5M for 24 h.

Vickers mechanical strength measurements have shown that the metal surface hardness is maintained throughout the process and that this property is conserved even after several months. In the case of aluminum, a non-significant increase in the hardness properties was observed after the etching process. Kim et al. also described such increase in mechanical strength of porous aluminum according to the strength of the process. In titanium a slight decrease in the Vickers hardness values was observed. Similar results were shown by Furumoto et al. where significant decrease of Vickers hardness where observed depending on the increasing porosity of titanium. In our case, the smaller modifications of the hardness properties in both aluminum and titanium can be explained by the utilization of a less aggressive treatment. In the case of gold, unlike the other metals, a significant decrease in mechanical strength was observed post etching. Gold, unlike aluminum and titanium, has undergone a dealloying process. According to the EDS measurements, only the Au element was left in the surface after the gold treatment, making it softer than before the etching process where the material contained chemical elements that contributed to the hardness. In a similar way, Li et al. found that pure gold has a significant lower Vickers hardness value than gold alloys. These results confirmed high stability and mechanical durability of the porous materials over time even several months after their production. Moreover, the contact angle measurements allowed to assess an increase in the wettability of the samples after the etching process. This increase can be directly related to the formation of porous structures at the surface of the metal samples as previously shown.

In our mechanical deposition approach, nanoparticles were designed and fabricated to fit the pore size on the metal to allow proper entrapment. The high surface area of porous materials also improved the loading capacity of the metal to absorb the nanoparticles. As a demonstration for our method thymol odor molecules were encapsulated in polymer nanoparticles. The challenge of encapsulating odor molecules has been known in everyday consumables such as cleaning products, pesticides, perfumes, textiles, toiletries and more. In these regards, the insertion of nanoparticles in porous structure of the metals, by itself, has an advantage compared to other methods of deposition. The incorporation of nanoparticles does not affect the external appearance of the metal which is a considerable important when aesthetic parameters are accounted for. Several formulation methods ensure the stability of volatile molecules while minimizing their premature release. This formulation includes a variety of techniques such as emulsion solvent evaporation, emulsion electrospinning, electrospraying, spray-drying, lipid-based carriers, colloidal self-assembly, and molecular odorant binding. Here, even if all the porous metals can absorb nanoparticles, the release kinetics were performed using polymeric PLGA nanoparticles in porous titanium prepared using an emulsion solvent evaporation method.

Blind odor tests performed by volunteers revealed an efficient smell perception lasting 10 days in metals incorporated with thymol-loaded PLGA nanoparticles. Moreover, it was found that the intensity of the thymol smell was higher in PLGA nanoparticle-containing metals in comparison with the free thymol-covered metal surfaces, despite the equal total quantities of thymol that were deposited in each sample surface. These results show that the PLGA nanoparticles enhance the sense of smell over a longer time. This result requires further confirmation, and it may offer future research perspectives in the field of odor perception.

Release studies of thymol from the nanoparticles deposited in porous titanium showed a chemical retention of almost 40% after 15 days and odor retention for 10 days. This is possibly due to the special properties of the encapsulation material, PLGA, which is a biodegradable polymer that decomposes when exposed to water. In our case, because the product is dry, it is probable that the degradation is negligible for many weeks and that it is possibly slightly affected by the humidity of the air, and that the main mechanism to release the entrapped compound is controlled by diffusion through the polymer chain network. Similar attempts to enhance smell retention have shown 40% volatile retention after 10 days from chitosan nanoparticles and 50% after 15 days from gelatin-Arabic gum. However, encapsulating volatile molecules on dry surfaces for longer periods remains challenging. Odor release depends on several parameters, such as the air/liquid interface release, the encapsulation technique, the material type, and the odor molecule used. Unfortunately, few studies have compared different release conditions to identify the specific parameters required to improve retention. In the future, a possible method to modify diffusion efflux is to add a thin polymer membrane or use polymers with higher molecular weight.

The blind odor tests and the HPLC measurements correspond to the preliminary studies required in the cosmetic and perfume industry to assess the release kinetics of different odor formulations. In the future, additional analytical methods should be considered, such as Gas Chromatography-Mass Spectrometry (GC-MS) and Solid-Phase Microextraction (SPME), to measure the concentration of odor molecules released in the air over time. Furthermore, safety and regulatory tests should be performed to assess that the technology does not cause any toxic effects in contact with the body.

Finally, a titanium ring was created using advanced metal 3D printing techniques. The external visualization of the object was unaffected by the etching and particle deposition processes. This jewel prototype could be potentially utilized as a portable system for porous materials that release odors. The described prototype provides a proof of concept rather than a commercially-ready technology and can serve as a starting point for future products and for additional similar odor-releasing objects or devices. Titanium is also frequently employed in other fields such as the biomedical sector; therefore, this innovation, based on nanoparticles released from porous structures, should be considered in this field as well.

1.-50. (canceled)

51. An object having a metallic surface with a chemically etched surface porosity comprising a plurality of etched surface nanopores, said plurality of nanopores being of a predefined porosity profile and containing polymeric nanoparticles.

52. The object according to claim 51, wherein the nanopores having an average size between 10 and 500 nm.

53. The object according to claim 51, wherein the nanoparticles are provided solely in the chemically etched nanopores.

54. The object according to claim 51, wherein the chemically etched surface comprising the nanopores containing nanoparticles is not provided with an external coating; or is provided with a degradable or biodegradable external coating.

55. The object according to claim 54, wherein the external coating is formed of a degradable or a biodegradable material selected amongst cellulose, starch, polyhydroxyalkanoates, polylactide, polycaprolactone, collagen, polyesters, polycarbonates, and polypeptides.

56. The object according to claim 51, wherein the nanoparticles are selected from nanocarriers, nanospheres, nanocapsules, core/shell nanostructures, micelles, and matrix nanostructures formed of a matrix material.

57. The object according to claim 56, wherein the nanoparticles are selected to modulate a mechanical, physical, chemical or a functional property of the metallic object or surface.

58. The object according to claim 51, wherein the nanoparticles are selected from:
nanoparticles of a first polymeric material encapsulating or containing a second material;
polymeric nanoparticles of a degradable or biodegradable polymer containing at least one active material selected from drugs, cosmetic materials, fragrant materials, coloring materials, flavoring materials, antimicrobial materials, insecticidal materials, insect-repelling materials, agriculturally acceptable materials, wherein the at least one active material is released from the nanoparticles over time;
polymeric nanoparticles of a degradable or biodegradable polymer containing at least one active material selected from drugs, cosmetic materials, fragrant materials, coloring materials, flavoring materials, antimicrobial materials, insecticidal materials, insect-repelling materials, agriculturally acceptable materials, wherein the at least one active material is to be released from the nanoparticles over time, and wherein the degradable or biodegradable polymer is a functional material.

59. An object according to claim 51 having a metallic surface with a plurality of nanopores provided in a pore pattern formed by chemical etching of the metallic surface, the plurality of chemically etched nanopores comprising, holding, docking or hosting polymeric nanoparticles comprising at least one pharmaceutical, drug or active agent, wherein the polymeric nanoparticles are configured to degrade or biodegrade overtime to bring about release of the at least one pharmaceutical, drug or active agent.

60. The object according to claim 59, wherein the at least one pharmaceutical, drug or active agent is a material used in medicine or veterinary.

61. The object according to claim 59, being an implant or a medical device positioned against a tissue or an organ of a human or animal subject.

62. An object according to claim 51 having a metallic surface having a plurality of nanopores provided in a pore pattern formed by chemical etching of the metallic surface, the plurality of chemically etched nanopores comprising, holding, docking or hosting polymeric nanoparticles comprising at least one material, wherein the polymeric nanoparticles are configured to degrade or biodegrade overtime to bring about release of the at least one material.

63. A process for manufacturing an object according to claim 51, the process comprising contacting a metallic surface of an object having a plurality of surface nanopores with a medium comprising polymeric nanoparticles and allowing said nanoparticles to occupy at least a portion of the plurality of nanopores, wherein the surface nanopores have been formed by chemical etching.

64. The process according to claim 63, the process comprises treating the object to remove nanoparticles present outside of the nanopores.

65. The process according to claim 63, the process comprising obtaining the object having an etched metallic surface decorated with a plurality of nanopores.

66. The process according to claim 63, the process comprising etching a metallic surface of an object under conditions selected to form a plurality of surface nanopores having a predefined porosity profile.

67. The process according to claim 63, wherein the etchant is selected from Adler etchant (comprising copper ammonium chloride, HCl, FeCl₃, water), ammonium persulfate etchant (comprising ammonium persulfate, and water), aluminum AI-NaOH etchant (comprising NaOH and water), etchant commercially available under ASTM No. 30 (comprising ammonia, H₂O₂, and water), etchant commercially available under ASTM No. 97 (comprising KOH and water), etchant commercially available under ASTM No. 157 (comprising CrO₃, HCL and water), Berahas etchant (comprising Na₂S₂O₃, K₂S₂O₅ and water), Carpenters stainless steel etchant (comprising FeCl₃, CuCl₂, HCl, nitric acid, and ethanol), etchant commercially available under Copper No. 1 (comprising nitric acid and water), etchant commercially available under Copper No. 2 (comprising HCl, ferric chloride and water), dichromate etchant (comprising K₂Cr₂O₇, H₂SO₄, NaCl and water), Frys reagent (comprising HCl, copper chloride, water and ethanol), Inconel etchant (comprising nitric acid, HCl, H₂O₂ and water), Kalling's No. 2 (comprising CuCl₂, HCl, and ethanol), waterless Kalling's reagent (comprising CuCl₂, HCl, and ethanol), Kellers Etchant (comprising nitric acid, HCl, HF and water), Klemm's reagent (comprising sodium thiosulfate, potassium metabisulfite and water), Kroll's reagent (comprising nitric acid, HF and water), Nital (comprising ethanol and nitric acid), Marble's reagent (comprising CuSO₄, HCl, and water), Murakami's reagent (comprising K₃Fe(CN)₆, KOH and water), Oberhoffers reagent (comprising FeCl₃, SnCl₂, HCl, ethanol and water), Picral (comprising ethanol and picric acid), Ralphs etchant (comprising FeCl₃, CuCl₂, HCl, ethanol and water), Schantz etchant (comprising FeCl₃, H₂SO₄, HCl, nitric acid, acetic acid and water), V2A etchant (comprising HCl, HBr, nitric acid and water), Weck's etchant (comprising ammonium bifluoride, HCl and water), Winsteard's reagent (comprising picric acid, HCl, surfactant, and ethanol).

68. The process according to claim 63, wherein the etchant is an etching solution comprising HNO₃ or comprising NaOH.

69. The process according to claim 63, the process comprising etching a metallic surface of an object to produce therein a plurality of nanopores and contacting the etched surface with a medium comprising nanoparticles or with a powder comprising or consisting the nanoparticles to permit docking of at least a portion of the nanoparticles in the plurality of nanopores.

70. A process for manufacturing an object according to claim 51, the process comprising:
contacting an object having a metallic surface with an etchant to form a plurality of nanopores on the surface;
contacting the etched surface with a medium (solution, dispersion or emulsion) comprising polymeric nanoparticles or with a powder comprising or consisting the nanoparticles and allowing said nanoparticles to occupy at least a portion of the plurality of nanopores; and
optionally forming a protective coating or film of a degradable or biodegradable material on the metallic surface having been treated with the nanoparticles.
